(19) United States
(12) Patent Application Publication
(10) Pub. No.: US 2022/0304746 A1
(43) Pub. Date: Sep. 29, 2022

(54) MEDICAL DEVICES HAVING CONDUCTIVE JUNCTIONS

(71) Applicant: ECOM MEDICAL, INC., SAN JUAN CAPISTRANO, CA (US)

(72) Inventors: GUY RUSSELL LOWERY, SAN JUAN CAPISTRANO, CA (US); JOHN P. GREELIS, CARLSBAD, CA (US); GARY H. SANDERS, RANCHO SANTA MARGARITA, CA (US)

(21) Appl. No.: 17/625,070

(22) PCT Filed: Jul. 9, 2020
(86) PCT No.: PCT/US2020/041375

§ 371 Date: Jan. 5, 2022

Related U.S. Application Data
(60) Provisional application No. 62/871,926, filed on Jul. 9, 2019.

(51) Int. Cl. A61B 18/14; A61B 5/00; A61B 18/08; A61B 5/0538

(52) U.S. Cl. CPC A61B 18/1492; A61B 5/6853; A61B 18/082; A61B 5/0538; A61B 2018/00577

(57) ABSTRACT

A method for creating a conductive junction in a system for performing a diagnostic or therapeutic procedure includes placing a first elongate conductor through a first lumen of an elongate body, creating a hole in a wall of the elongate body adjacent the first lumen at a distal portion of the elongate body, applying an electrically conductive material to a portion of an outer circumference of the elongate body at the distal portion of the elongate body to form an electrode, and electrically coupling the first elongate conductor to the electrode.

### FIELD OF THE INVENTION

The field of the invention generally relates to systems for performing diagnostic or therapeutic procedures within a living body.

### BACKGROUND

A variety of medical devices are manufactured and utilized which incorporate conductors, sensors, electrodes, circuits, or other electrical elements on elongate shafts or tubing. These medical devices are used in diagnostic and/or therapeutic procedures, in which it is desired to carry a signal either toward the patient or away from the patient along the elongate shaft of the medical device. In many cases, the diameter or transverse dimension of the shaft must be as small as possible, so that it may better fit through a natural or medically-created orifice in the body of the patient, or fit down a natural or medically-created space in the body of the patient. In addition, the medical devices are expected to reliably carry signals, regardless of the shape they take within the patient, or the stresses that are placed upon them.

### SUMMARY OF THE INVENTION

In one embodiment of the present disclosure, a system for performing a diagnostic or therapeutic procedure within a subject includes a device configured for insertion within a lumen or duct of the subject, the device including an elongate body having a longitudinal axis, a proximal end, and a distal end, one or more lumens extending within the elongate body, a first conductor carried within at least one or more lumens, the first conductor having a proximal end and a distal end, an electrode carried on an exterior of the elongate body and electrically coupled to the distal end of the first conductor, and a connector having a first end configured to couple to an input of a control console configured for controlling operation of the device, and a second end electrically coupled to the proximal end of the first conductor.

In another embodiment of the present disclosure, a system for performing a diagnostic or therapeutic procedure within a subject includes a device configured for insertion within a lumen or duct of the subject, the device including an elongate body having a longitudinal axis, a proximal end, and a distal end, a plurality of conductors embedded within the elongate body, an electrode carried on an exterior of the elongate body and electrically coupled to at least one of the plurality of conductors, and a connector electrically coupled to the at least one of the plurality of conductors.

In yet another embodiment of the present disclosure, a method for creating a conductive junction in a system for performing a diagnostic or therapeutic procedure includes placing a first elongate conductor through a first lumen of an elongate body, creating a hole in a wall of the elongate body adjacent the first lumen at a distal portion of the elongate body, applying an electrically conductive material to a portion of an outer circumference of the elongate body at the distal portion of the elongate body to form an electrode, and electrically coupling the first elongate conductor to the electrode.

### BRIEF DESCRIPTION OF THE DRAWINGS

FIG. 1 is an elevation view of an elongate medical device according to an embodiment of the present disclosure.

FIG. 2 is a detail view of the elongate medical device of FIG. 1 taken within circle 2.

FIG. 3 is a detail view of the elongate medical device of FIG. 1 taken within circle 3.

FIG. 4 is a cross-sectional view of the elongate medical device of FIG. 2 taken along line 4.

FIG. 5 is a cross-sectional view of the elongate medical device of FIG. 3 taken along line 5.

FIG. 6 is a top view of the elongate medical device of FIG. 2.

FIG. 7 is a bottom view of the elongate medical device of FIG. 2.

FIG. 8 is a detail view of a conductive joint site of the elongate medical device of FIG. 6 taken within circle 8.

FIG. 9 is a view of the conductive joint site of FIG. 8 after a filing operation.

FIG. 10 is a view of the conductive joint site of FIGS. 8 and 9 after the application of an electrode.

FIG. 11 is a perspective view of an electrical connector junction of the elongate medical device in a first state of assembly.

FIG. 12 is a perspective view of the electrical connector junction in a second state of assembly.

FIG. 13 is a perspective view of the electrical connector junction in a third state of assembly.

FIG. 14A is a perspective view of the electrical connector junction in a fourth state of assembly.

FIG. 14B is a perspective view of an alternative electrical conductor junction, according to an embodiment of the present disclosure.

FIG. 15 is a cross-sectional view of the elongate medical device of FIG. 1 taken along line 15.

FIG. 16 is an elevated view of a conductive wire, according to an embodiment of the present disclosure.

FIG. 17 is cross-sectional view of the elongate medical device of FIG. 9 taken along line 17.

FIG. 18 is cross-sectional view of the elongate medical device of FIG. 1 taken along line 18.

FIG. 19 is a system for cardiovascular sensing including a laryngeal mask, according to an embodiment of the present disclosure.

FIG. 20 is a partial sectional view of the laryngeal mask of FIG. 19 in place within a subject.

FIG. 21 is a partial sectional view of a system for cardiovascular sensing, according to another embodiment of the present disclosure.

FIG. 22 is perspective view a system for cardiovascular sensing including a sensing device having an expandable member, according to an embodiment of the present disclosure.

FIG. 23 is a perspective view of a system for sensing electrical activity of the heart according to an embodiment of the present disclosure.

FIG. 24 is a view of a sensing device placed within the trachea and a bronchus of a subject according to an embodiment of the present disclosure.

FIG. 25 is perspective view a system for cardiovascular sensing including a sensing device, according to an embodiment of the present disclosure.

FIG. 26 is a partial sectional view of the sensing device of FIG. 25 within an esophagus of a subject in a low-profile state, according to an embodiment of the present disclosure.

FIG. 27 is a partial sectional view of the sensing device of FIG. 25 within an esophagus of a subject in an expanded state, according to an embodiment of the present disclosure.

FIG. 28 is a perspective view of a tubular component of a medical device according to an embodiment of the present disclosure.

FIG. 29 is a perspective view of a tubular component of a medical device according to an embodiment of the present disclosure.

FIG. 30 is an elevated view of a tubular component of a medical device according to an embodiment of the present disclosure.

FIG. 31 is a schematic representation of a first embodiment of a medical device according to the present disclosure.

FIG. 32 is a schematic representation of a second embodiment of a medical device according to the present disclosure.

FIG. 33 is a sectional view of an alternative embodiment of a conductive joint site, according to an embodiment of the present disclosure.

FIG. 34 is a cross-sectional view of an elongate tube, according to another embodiment of the present disclosure.

### DETAILED DESCRIPTION OF THE ILLUSTRATED EMBODIMENTS

One shortcoming that limits the ability to manufacture a medical device incorporating multiple sensors, electrodes and/or circuits (e.g., for monitoring or treating a patient) is that there are limits to the number of circuit wires and/or electrically conductive tracings (also called traces) that can be utilized for a particular size of a device. This is evident on devices that are intended to be introduced into the body where a natural lumen such as a vein, artery, trachea or esophagus will be utilized. As an example, a nasal gastric tube is introduced into the stomach through the nose and down the descending esophagus. The tube often being approximately 4 mm in diameter, it is difficult to print/deposit or place linear wires/conductive tracings on the surface to connect with sensors. Because there are often additional current and isolation requirements for the device, one may only be able to deposit three or four conductive tracings on the surface of the device, when more may be needed or desired. The present disclosure overcomes these limitations while having minimal or no impact on device diameter.

This concept can also be applied in various geometric configurations including flat or other configurations and in flexible, inflatable or rigid formats dependent upon desired function.

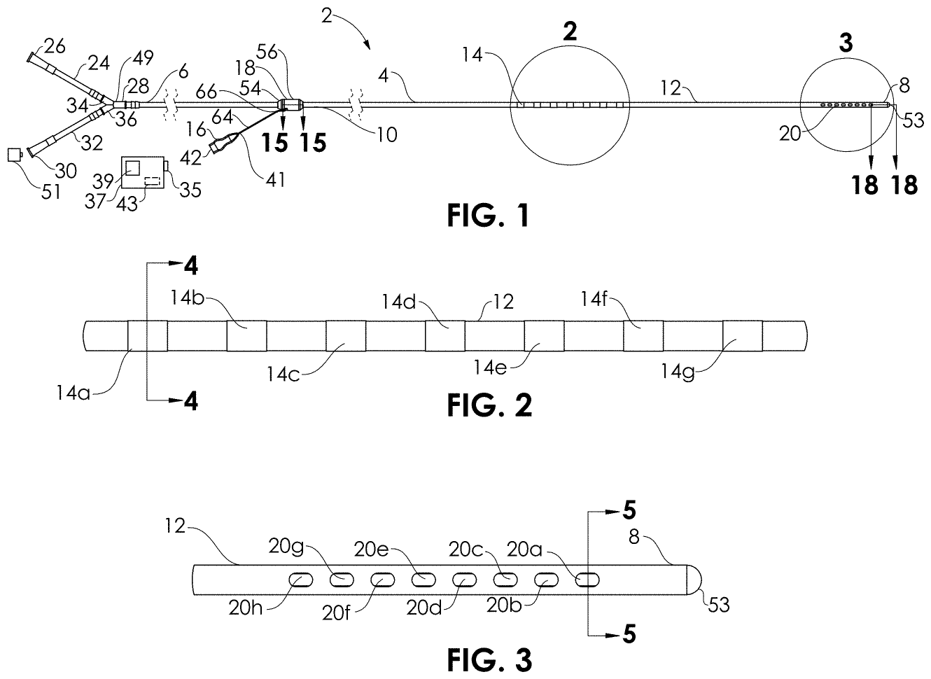

FIGS. 1-3 illustrate an elongate medical device 2 in the form of a nasogastric tube (NG tube). A NG tube can be used for feeding, administration of drugs, or aspiration of gastric contents. Special NG tubes may also include sensing or energy delivery capabilities which may make use of circuitry and conductive wires or conductive traces, within or on the NG tube itself. The medical device 2 includes an elongate tube 4 having a proximal end 6 and a distal end 8. The elongate tube 4 has an insertable portion 12 bounded by the distal end 8 and a proximal insertable end 10. The insertable portion 12 may be sized for insertion into a duct or opening in a patient according to each particular application. In some embodiment the insertable portion is between about 30 cm and about 150 cm, or between about 50 cm and about 100 cm, or about 70 cm. The elongate tube 4 may have a diameter or (if nor circular) a maximum transverse dimension of between about 0.5 mm and about 40 mm, or between about 1 mm and about 30 mm, or between about 2 mm and about 15 mm, or between about 4 mm and about 8 mm, or about 6 mm. The elongate tube 4 may comprise a polymeric material, such as polyvinyl chloride, nylon, polyurethane, or polyether block amide. The polymeric material may have a durometer of between about 65 A and 95 A, or about 70 A and 90 A, or about 80 A (shore). The polymeric material may include radiopaque doping, or the elongate tube 4 may itself include a radiopaque stripe or band. Radiopaque materials may include tantalum, barium sulfate, platinum, gold, or platinum alloys. The distal end 8 includes a blunt tip 53, that may comprise a hemispheric portion of a similar material, which may be adhesively bonded with urethane or UV-curable adhesive, epoxy bonded, or thermally bonded by hot air, infrared heating, ultrasonic welding, or even solvent bonded. Seven electrodes 14a-g are linearly arrayed on the insertable portion 12 of the elongate tube 4, and are electrically coupled to a connector 16 via a coupling 18. The connector 16 may have a series of pins 42 that are configured to plug into an input 35 of a console 37 configured for control and/or communication via a user interface 39. A multiwire cable 64 is electrically connected to the connector 16 at one end 41 and to the coupling 18 at the other end 66. The seven electrodes 14a-g may comprise six “sense” electrodes, and one ground electrode. Multiple electrodes can add reliability to devices. Any one of the electrodes 14a-g may be assigned as a ground. Thus, only two electrodes are required to contact body tissue at any one time in order to obtain at least one measurement. In certain anatomical conditions, in which certain electrodes cannot effectively contact body tissue, a measurement can still be made with at least two electrodes contacting.

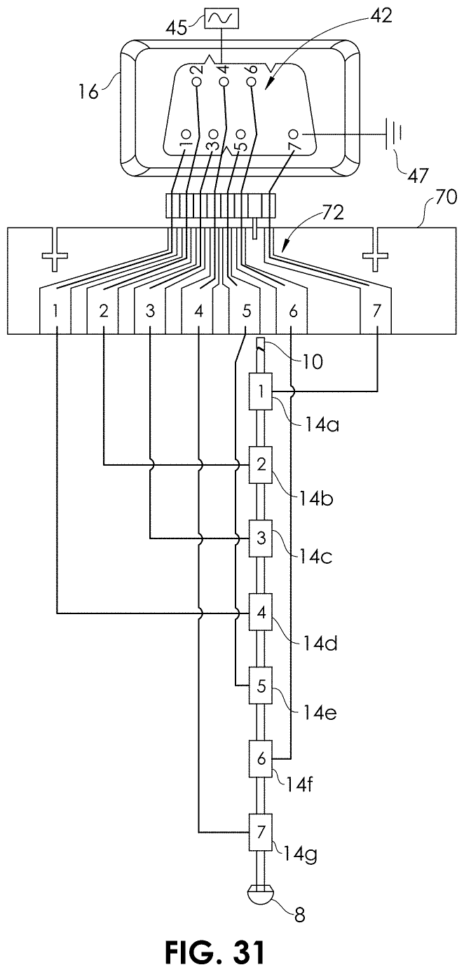

In some embodiments, one of the outer (bookend) electrodes 14a, 14g may serve as a ground electrode, while the other of the outer (bookend) electrodes 14a, 14g may serve as an excitation electrode, and one or more of the interior (book) electrodes 14b, 14c, 14d, 14e, 14f may serve as the sense electrodes. The medical device 2 may be operated by a console 37 (having a controller 43, microcontroller, etc.) that actively identifies, in use, which of the electrodes (e.g., 14b, 14c, 14d, 14e, 14f) has/have an available signal, and then uses the signal(s) from only one or more of those particular electrodes. An excitation signal (e.g. sinusoidal voltage) is applied to the excitation electrode, and the sense electrodes each are configured to measure a sensed signal from the tissue they each contact. In a first embodiment, as illustrated in FIG. 31, electrode 14a is configured to be utilized as a ground electrode, while electrode 14g is configured to be utilized as an excitation electrode, and electrodes 14b, 14c, 14d, 14e, 14f are each configured to be utilized as sense electrodes. Pin 42 number “7” of the connector 16 is configured to be connected to ground 47 (e.g., via the console 37), and pin number “4” of the connector 16 is configured to be connected to a sinusoidal voltage/current input 45 (e.g., via circuitry of the console).

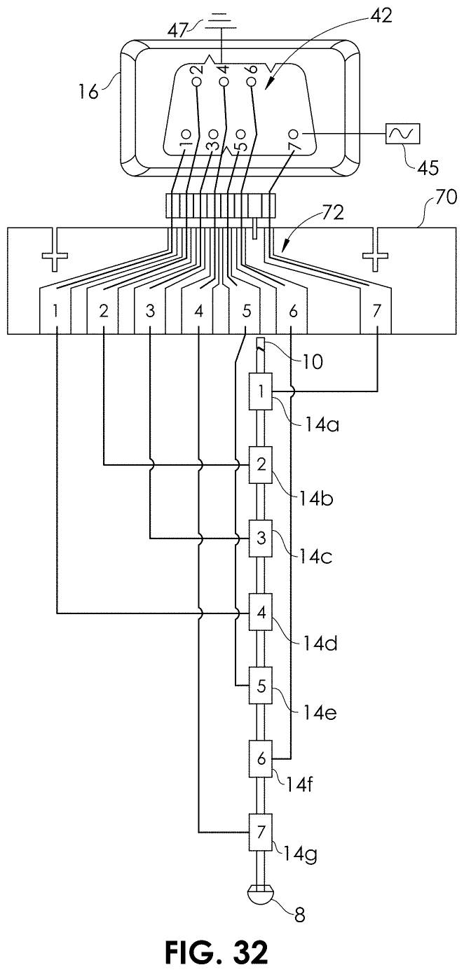

In a second embodiment, as illustrated in FIG. 32, electrode 14g is configured to be utilized as a ground electrode, while electrode 14a is configured to be utilized as an excitation electrode, and electrodes 14b, 14c, 14d, 14e, 14f are configured to be utilized as sense electrodes. Pin 42 number “4” of the connector 16 is configured to be connected to ground 47 (e.g., via the console 37), and pin number “7” of the connector 16 is configured to be connected to a sinusoidal voltage/current input 45 (e.g., via circuitry of the console). An electrical strip 70 having electrically conductive tracings 72, shown in FIGS. 31 and 32, will be described in more detail in the description to follow. The connections between specific pins 42 (p-1, 2, 3, 4, 5, 6, 7) of the connector 16 to conductive tracings 72 (t-1, 2, 3, 4, 5, 6, 7) to electrodes 14a-14g (e-1, 2, 3, 4, 5, 6, 7) is listed in Tables 1 and 2.

TABLE 1
First Embodiment, FIG. 31

| Connector Pin | Electrical Strip Tracing | Electrode |
|---|---|---|
| 1 | 1 | 4 |
| 2 | 2 | 2 |
| 3 | 3 | 3 |
| 4 | 4 | 7 |
| (AC input) | | |
| 5 | 5 | 5 |
| 6 | 6 | 6 |
| 7 | 7 | 1 |
| (Ground) | | |

TABLE 2
Second Embodiment, FIG. 32

| Connector Pin | Electrical Strip Tracing | Electrode |
|---|---|---|
| 1 | 1 | 4 |
| 2 | 2 | 2 |
| 3 | 3 | 3 |
| 4 | 4 | 7 |
| (Ground) | | |
| 5 | 5 | 5 |
| 6 | 6 | 6 |
| 7 | 7 | 1 |
| (AC input) | | |

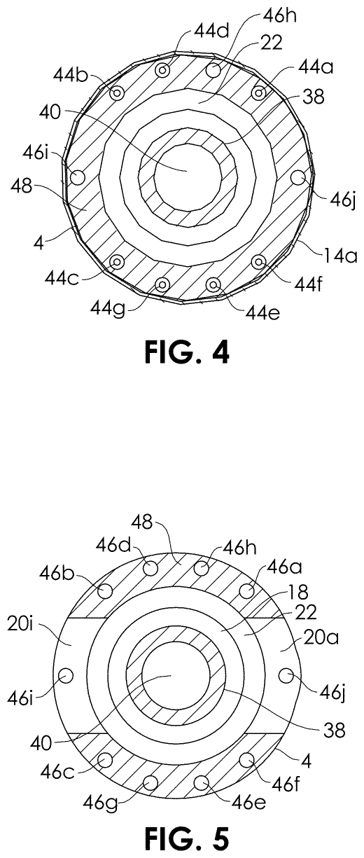

Sixteen luminal ports 20a-p (eight on each side) extending through a wall 48 in the elongate tube 4 allow access to a longitudinally extending internal lumen 22 (FIGS. 4-5). The internal lumen 22 extends through the elongate tube 4 and is secured to a distal end 49 of a connector 28 at the proximal end 6. The internal lumen 22 may have an inner diameter of between about 0.055 inch (1.4 mm) and about 0.425 inch (10.8 mm), or between about 0.113 inch (2.8 mm) and about 0.226 inch (5.74 mm), or about 0.170 inch (4.3 mm). The connector 28 is fluidly coupled to an injection/aspiration port 26 via an extension tube 24, which extends from a first branch 34 of the connector 28 to the port 26. The injection/aspiration port 26 may comprise a female luer connector, and is configured to attach to a syringe, vacuum pump, or a tubing set. A vent port 30 also extends from a second branch 36 of the connector 28, via an extension tube 32. The vent port 30 may also comprise a female luer connector. The vent port 30 and extension tube 32 are fluidly coupled to a vent tube 38 (FIGS. 4-5) having an inner lumen 40, the vent tube 38 extending through the internal lumen 22 of the elongate tube 4 to the distal end 8. The vent tube 38 allows air to be pulled into the vent port 30 and extension tube 32 when a vacuum (negative pressure) is applied to the injection/aspiration port 26, thus aiding aspiration and avoiding collapse of the internal lumen 22 in some cases. A luer cap 51 may be sealingly placed onto the vent port 30 when desired, to stop air from being pulled into the vent port 30. Turning to FIG. 18, the vent tube 38 may include a distal skive 98 which is angled to maintain flow patency, or otherwise help to avoid suctioning or clogging against internal features of the elongate medical device 2. A fillet 99 serves to minimize the possibility of punctures in the wall 48 of the elongate tube 4 or in the blunt tip 53, by reducing the sharpness of the skive 98. Alternatively, or additionally, the vent tube 38 may include one or more optional sideholes 55, to further preserve flow patency. The vent tube 38 may comprise metallic or polymeric materials, such as PVC, and may have an inner lumen 40 diameter of between about 0.75 mm and about 3 mm, or between about 1 mm and about 2 mm. Seven electrical wires 44a-g extend within seven circumferentially-arrayed wire lumens 46a-g which each extend within the wall 48 surrounding the internal lumen 22. The electrical wires 44a-g comprise copper wires and are each pulled through one of the lumens 46a-g when the medical device 2 is assembled. The copper wire may have a diameter of between about 0.002 inch (0.05 mm) and about 0.008 inch (0.20 mm), or between about 0.003 inch (0.076 mm) and about 0.006 inch (0.152 mm). The wire may be single strand or may comprise two or more strands. In alternative embodiments, the electrical wires 44a-g may be combined with the elongate tube 4 during an overextrusion or co-extrusion process. In other alternative embodiments, an electrically conductive adhesive or epoxy may be injected down the lumens 46a-g and allowed or made to cure, in order to create elongate conductive elements that are analogous to standard wire conductors. The injected material would be able to substantially fill the lumens 46, and thus the lumens could be sized efficiently, with little or no wasted space. No insertion of wires would thus be needed. Additional lumens 46h, 46i, 46j may also extend longitudinally within the wall 48 of the tube 4, in case additional wires are needed, or to aid in a symmetric or balanced, unwarped tubing extrusion. These lumens 46h, 46i, 46j may also be used to fit other elongate elements, such as a thermocouple, a thermistor, an optical fiber, an optical fiber bundle, an ultrasonic element, or a pressure sensor.

FIG. 34 illustrates an alternative embodiment of an elongate body tube 21 having a configuration which eliminates the separate vent tube 38 of FIG. 18. The body tube 21 includes several lumens 23a-h which are analogous to the lumens 46 described in the embodiment of FIGS. 4-5, and an internal lumen 25 which is analogous to the internal lumen 22. A vent lumen 27, co-extending with the lumens 23a-h and the internal lumen 25 within the elongate body tube 21, is configured to function in a similar manner to the inner lumen 40 of the vent tube 38. A reduced total diameter of the body tube 21 may be achieved, because there is only one additional wall 29 required to create the feature of the vent lumen 27, instead of the walls on both sides in the vent tube 38. The removal of the thickness of this additional wall makes for a smaller diameter, and thus, more flexible body tube 21, which is thus made more appropriate for pediatric patients. In some embodiments, for visualization on fluoroscopy, a longitudinally-running radiopaque stripe 31 may be co-extruded onto the body tube 21. The radiopaque stripe 31 may comprise a polymer similar to the polymer of the rest of the body tube 21, but it may be additionally doped with a radiopaque material such as barium sulfate, tungsten, bismuth trioxide, bismuth subcarbonate, or bismuth oxychloride. Additionally, or alternatively, the stripe 31 may include colorant, such as titanium dioxide, for clearer viewing of the body tube 21 outside of the patient. The radiopaque stripe 31 not only allows the body tube 21 to be visualized inside the patient, but may also allow the visualization of the particular orientation of the body tube 21, either its rotational orientation or its amount of flexure.

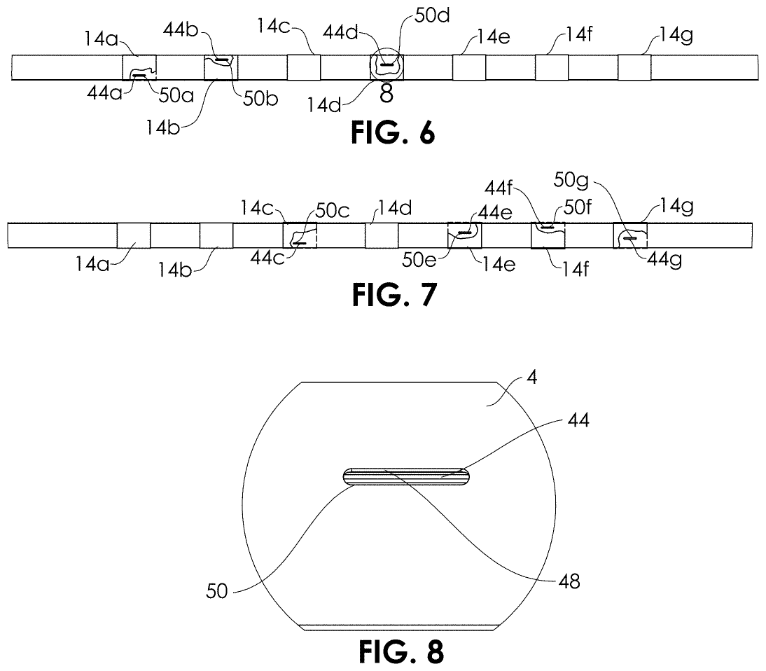

Returning to FIGS. 6-8, the electrodes 14a-g are connected to the electrical wires 44a-g by removing a portion of the wall 48 of the tube 4 that is radially outward from the lumens 46a-g, thus exposing each of the wires 44a-g. Thus, a window 50 in the wall 48 of the tube 4 is created for each of the seven lumens 46a-g. Each of the windows 50a-g is located at a different longitudinal location on the tube 4. In a top view in FIG. 6, windows 50a, 50b, and 50d are visible, while in a bottom view in FIG. 7, windows 50c, 50e, 50f, and 50g are visible. As shown in FIG. 8, the window 50 can be an elongate opening, including a rectangular or oval opening in a portion of the wall 48. The window 50 may be slit, skived, die cut, shaved or thermally created, in order to expose the interior of the lumen 46 and the wire 44 inside.

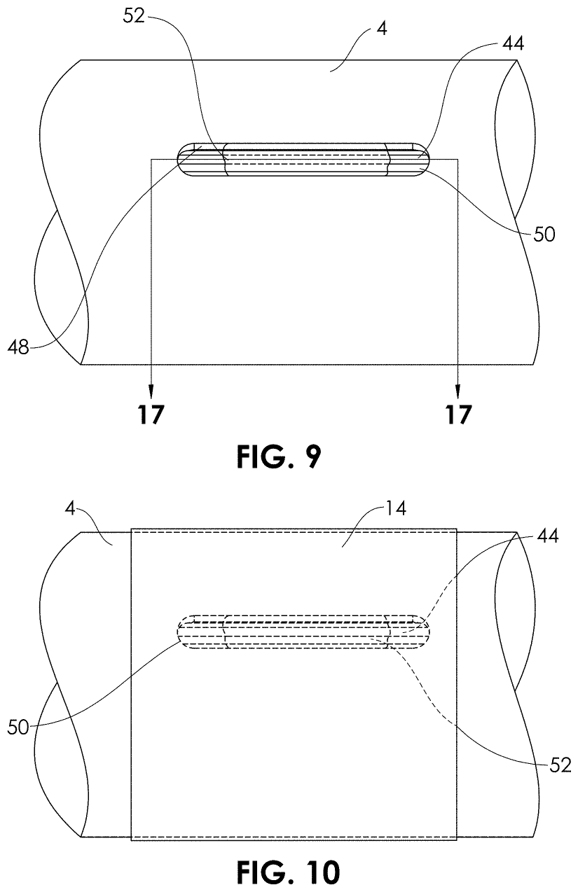

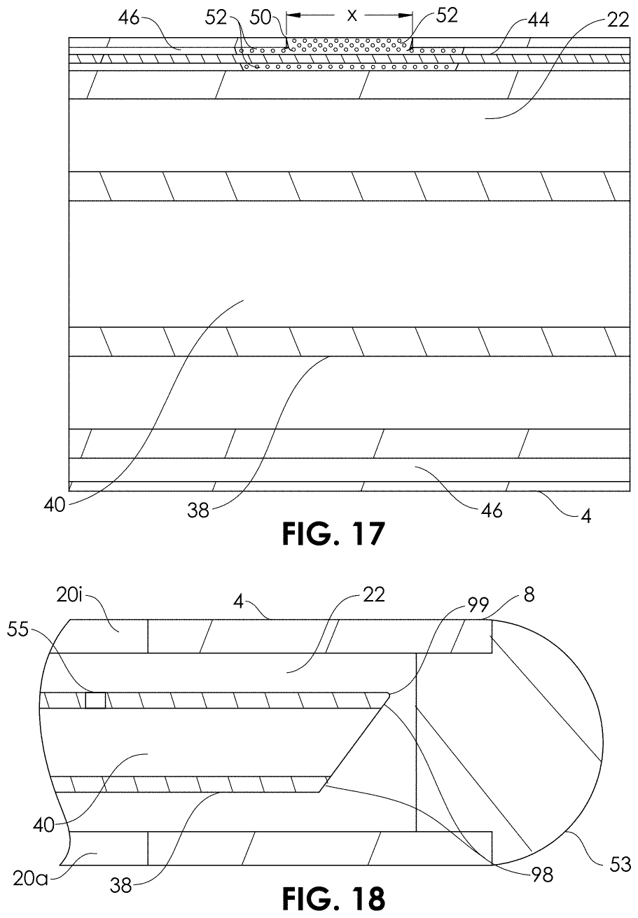

Turning to FIG. 9, after the window 50 is created, an electrically conductive epoxy 52 is applied within at least a portion of the window 50, so that it wets the wire 44. The wire 44 may be cleaned or abraded and cleaned prior to the application of the conductive epoxy 52, to aid the engagement between the conductive epoxy 52 and the wire 44. It may be desired to apply enough of the conductive epoxy 52 so that it rises within the window 50 to generally match the total diameter of the tube 4. The conductive epoxy 52 is then allowed to cure, and, in some embodiments, the curing of the conductive epoxy 52 may be accelerated by the application of heat or ultraviolet energy. FIG. 17 illustrates one embodiment in which the conductive epoxy 52 is applied within the lumen 46 such that it substantially surrounds the wire 44. In some embodiments, the luminal diameter or transverse dimension may have enough clearance on both sides of the wire 44, to allow the conductive epoxy 52 to sufficiently surround the wire 44, and to be easily injected (depending on the viscosity of the conductive epoxy 52). In other embodiments, the conductive epoxy 52 may be applied such that it only wets one side of the wire 44 or an incomplete circumference of the wire 44 (e.g., 90°, 120°, 180°, 270°). The conductive epoxy 52 may comprise GPC-251A/B-2612 by Creative Materials of Ayer, Mass., USA, which is a two-part epoxy capable of room temperature cure. In some embodiments, an elevated temperature cure epoxy may be utilized, using a heat gun, an oven, infra-red heating, or other methods. Other conductive epoxies include Flexible-silver 17 by Epoxy International of Fort Lauderdale, Fla., USA. A clearance of about 0.003 inch to 0.006 inch (annulus thickness) around the wire 44 may be used, and the diameter of the lumens 46 may be sized accordingly, in relation to wire diameter or transverse dimension. In some embodiments, the lumens 46 may have a diameter or transverse dimension of about 0.010 inch to about 0.020 inch, or about 0.012 inch to about 0.018 inch, or about 0.014 inch to about 0.016 inch. The longitudinal dimension x of the window 50 may be between about 0.040 inch and 0.080 inch.

In FIG. 10, the electrode 14 is applied onto the tube 4 so that it creates an electrical contact with the conductive epoxy 52. Thus, at least indirectly, the electrode 14 makes an electrical contact with the wire 44. The electrode 14 may comprise a conductive epoxy similar or identical to the conductive epoxy 52 applied within the window 50. The electrode may be applied using a process and an application apparatus as disclosed in co-owned U.S. patent application Ser. No. 16/640,338, a national stage application of PCT/US18/47152 filed Aug. 21, 2018, published as WO2019/040393 Feb. 28, 2010, and titled “SYSTEMS AND METHODS FOR APPLYING MATERIALS TO MEDICAL DEVICES,” which is hereby incorporated by reference in its entirety for all purposes. The electrode 14 and/or the conductive epoxy 52 may include a conductive ink, and may alternatively comprise a conductive adhesive, including, but not limited to a UV-curable adhesive. The electrode 14 of the conductive epoxy 52 may comprise silver.

An alternative conductive joint site 11 is illustrated in FIG. 33, wherein two windows 13, 15 are cut or otherwise created in wall 48 of the tube 4. The one end 19 of the wire 44 is pulled out of window 13 and then reinserted through window 15 back into lumen 46. The wire 44 is bonded in place with an adhesive 17, which may comprise a UV curable adhesive, which is then cured via exposure to a UV-light for an appropriate amount of time. Other adhesives or epoxies may be used, including heat-accelerated-cure products. In some embodiments, only a small length of the wire end (e.g., distal end 19) need be “tucked” back into the lumen 46. For example, in some embodiments, the total tucked-in length LL may be between 2 mm and 5 mm. Additional adhesive 17 may be applied distally to the end 19 of the wire 44 (for example, to the right of the end 19 in FIG. 33), in order to completely seal off the lumen 46 distal to the wire 44. This sealing protects any capillary action of fluid (saline, medicants, water, body fluids, etc.) from contacting the wire 44, and thus protects against any potential short circuiting. A small hypodermic needle may be inserted into the window 15 and the adhesive 17 may be injected from the needle, distally, and allowed or made to cure. This may be done prior to the reinsertion of the wire end 19 into the lumen 46. As shown in FIG. 10, an electrode 14 is then applied onto the tube 4, in such a manner to create an electrical contact with the wire 44. Alternatively, conductive epoxy 52 may also be used as an intermediary element. The adhesive 17 or conductive epoxy 52 may include silver, or may include a conductive ink.

Turning to FIGS. 6-10, the distal ends of each of the wires 44a-g may terminate just distal of its respective window 50a-g, the windows 50a-g each filled with conductive epoxy 52a-g. Alternatively, the distal ends of each of the wires 44a-g may terminate substantially further distal, for example, adjacent the distal end 8 of the tube 4. This assumes that the lumens 46a-g extend substantially the entire length of the tube 4. In other embodiments, the lumens 46a-g may each terminate just distal of the respective windows 50a-g.

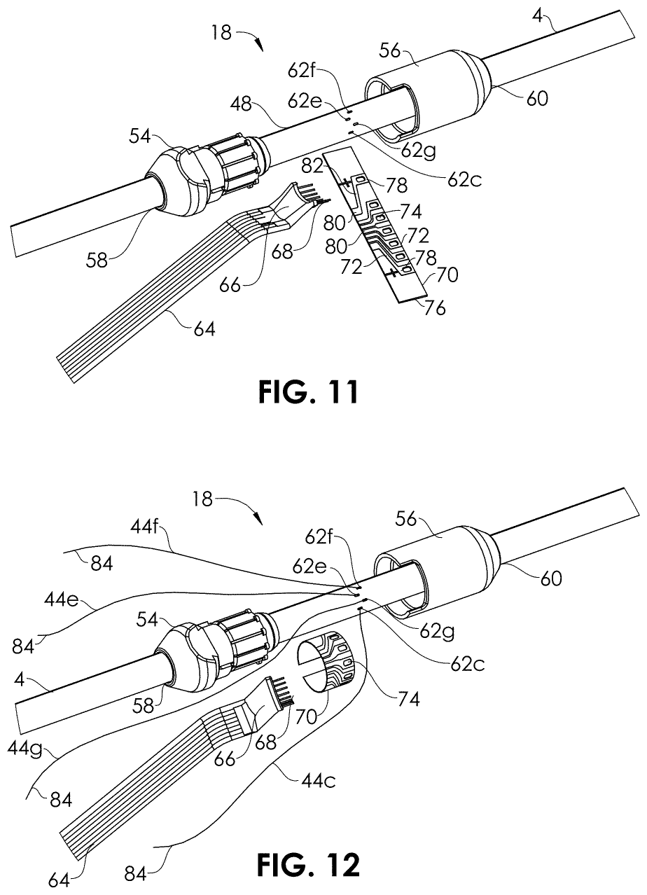

FIGS. 11-14A illustrate a process for assembling the coupling 18 (FIG. 1) of the elongate medical device 2. As shown in FIG. 11, a hub 54 having a bore 58 and a cover 56 having a bore 60 are inserted over the tube 4. Seven windows 62a-g are made in the wall 48 of the tube 4 for each of the seven lumens 46a-g. Each of the windows 62a-g is located over a different lumen 46a-g. Windows 62a, 62b, and 62d are not shown in FIGS. 11-14A, because they are on an opposite side of the tube 4. The multiwire cable 64 is also shown. A distal end 66 of the multiwire cable 64 includes stripped or uncoated conductive wires 68. An eight-wire multiwire cable 64 is shown, with one of the wires unused in the elongate medical device 2, However, other embodiments are contemplated which may use a multiwire cable 64 having different numbers of wires, for example between two and thirty, or between four and ten or between five and eight, or other quantities. An electrical strip 70 having conductive tracings 72 is shown in a flat, unrolled state in FIG. 11. The electrical strip 70 may comprise Kapton® (polyimide) sheet, and may have a thickness of between about 0.0005 inch and about 0.010 inch, or between about 0.003 inch and about 0.007 inch, or about 0.005 inch. In some embodiments, the polyimide layer of the strip 70 may have a thickness of about 0.0005 inch to about 0.0015 inch, and the conductive tracing 72 may have a thickness of about 0.001 inch and about 0.002 inch. The conductive tracing 72 may comprise copper, and may have a tin plating over the copper layer. The tin plating may by about 15% to about 35% of the thickness of the copper. A thin adhesive or primer layer may be included between the polyimide and the conductive material, and the polyimide may be pre-treated, mechanically, or chemically, for enhanced adhesion. The electrical strip 70 includes holes 74 through its wall 76, each surrounded by a portion of a conductive tracing 72. The conductive tracings 72 each include a first connection portion 78 surrounding each hole 74, a second connection portion 80, and a path 82 connecting the first connection portion 78 to the second connection portion 80. The second connection portions 80 are configured to be electrically coupled to the uncoated conductive wires 68 of the multiwire cable 64.

In FIG. 12, the electrical strip 70 is shown in the rolled or curved state into which it will be held in the final assembly. The width of the electrical strip (longitudinal dimension, when rolled) may be between about 20 mm and about 50 mm, or about 35 mm. Proximal portions 84 of the conductor wires 44a-g (44a, 44b, and 44d are not shown) are partially pulled out of their respective lumens 46a-g through the windows 62a-g. In some embodiments, each wire 44 is a single strand wire extending by itself through the lumen 46. In other embodiments, each wire 44 is a single strand wire that is folded in the middle of its length and doubled on itself, such that at one end is a 1800 bend 85 (FIG. 16) and the other end are the two strand ends 88, 90. For example, a 100 cm long strand would be used to make a 50 cm long doubled wire 44 in this case, having a first end 63 and a second end 65. One advantage of this folded, two-filar wire 44, is that a hook or other similar tool may be used to snare one or both of the filars of the wire 44 to pull it from the window 62, while the bend 85 assures that the two filars stay together, to facilitate assembly. Another advantage is that a more flexible overall device may be achieved. For example, two parallel 0.005 inch-diameter copper wires have about the same cross-sectional area as a single 0.007 inch-diameter copper wire, but they are significantly more flexible, even then next to each other. Another advantage is that two or more filar can be more reliable as a signal carrier, because of their redundancy; if one wire (filar) breaks, the other can nevertheless carry the signal.

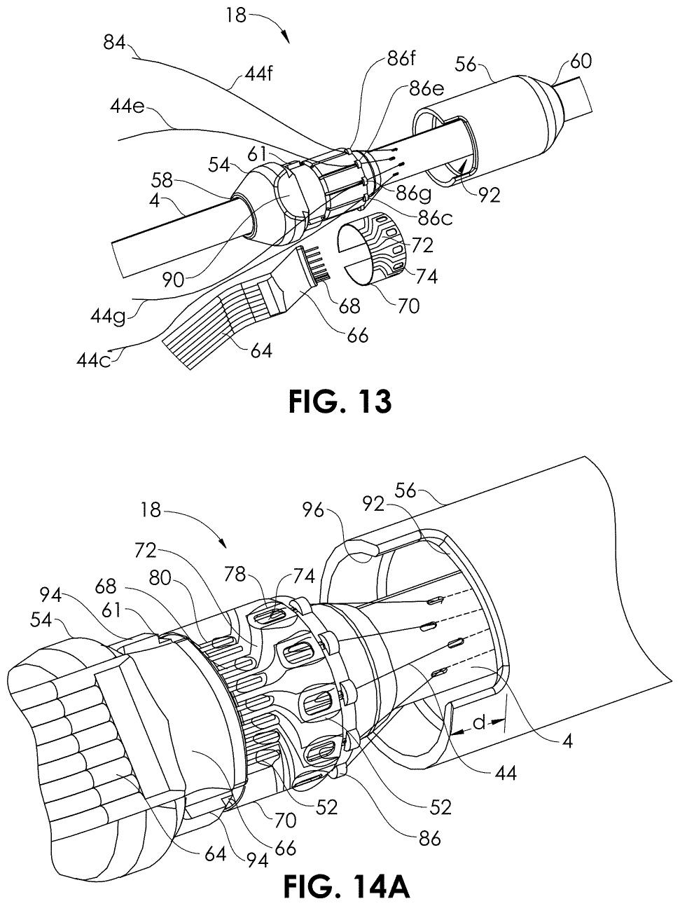

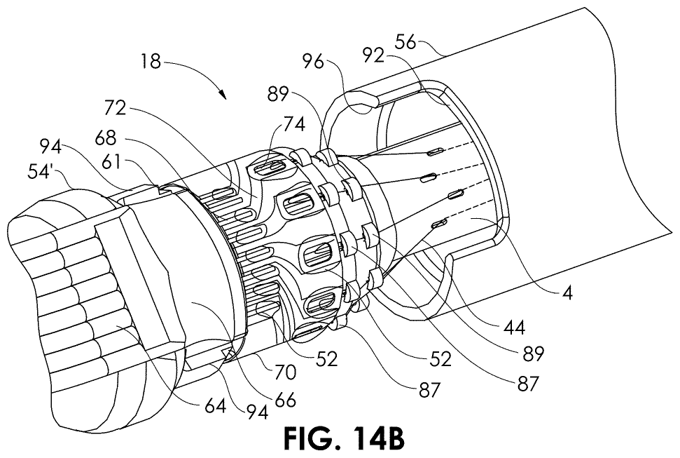

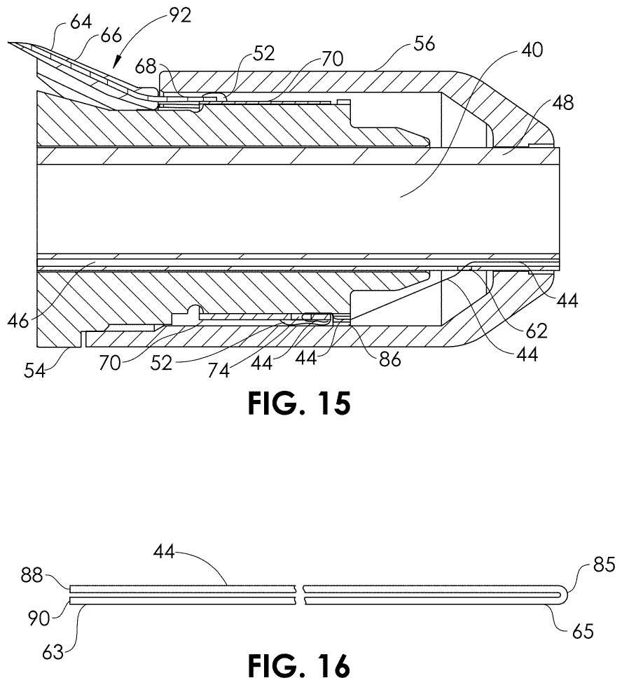

The uncoated conductive wires 68 are each attached to the second connection portions 80 and the proximal portions 84 of the conductor wires 44 are each attached to the first connection portions 78 of the conductive tracings 72 of the electrical strip 70, either while in its flat state or in its curved state, with conductive epoxy 52. See also, FIG. 14A. Alternatively, the uncoated conductive wires 68 may each be soldered to the second connection portions 80, and the proximal portions 84 of the conductor wires 44 may be soldered to the first connection portions 78 of the conductive tracings 72 of the electrical strip 70, either while in its flat state or in its curved state. Turning to FIG. 13, the proximal portions 84 of the wires 44 are pulled through distal loops 86a-g of the hub 54, in order to locate the wires 44 in their correct circumferential orientations for electrically connecting with the first connection portions 78 of the electrical strip 70. In FIG. 14A, the curved electrical strip 70 is shown in place extending circumferentially around a central portion 61 of the hub 54. The wires 44a-g are pulled through the respective holes 74 and bonded with conductive epoxy 52 to the first connection portions 78 of the conductive tracings 72 of the electrical strip 70, as shown in more detail in FIG. 15, that shows the cover 56 assembled over the hub 54. Thus, an electrically-isolated conductive path exists for each serial combination of connector pin 42/cable wire 68/conductive epoxy 52/tracing 72/conductive epoxy 52/wire 44/conductive epoxy 52/electrode 14. The distal end 66 (FIG. 13) of the multifilar cable 64 is configured to fit adjacent a flat 90 on the hub 54. The distal end 66 may even be bonded to the flat 90 with epoxy or adhesive. This arrangement maintains a lower profile and a secure fit. Once the connections are complete, the cover 56 can be snapped or bonded to the hub 54 to cover and protect the other components. FIG. 14A shows the assembly prior to placement of the cover 56. The cover 56 includes a relief 92 extending a longitudinal distance d and configured to accept the distal end 66 of the multifilar cable 64. One or more snaps 94 carried on the hub 54 are configured to snap the hub 54 into an undercut 96 within the cover 56. In some embodiments, the cover 56 may be unsnappable from the hub 54. The cover 56, in its fully closed state over the hub 54, is shown in FIG. 1. FIG. 14B illustrates an alternative hub 54′ having a first series of loops 87 and a second series of loops 89, instead of the single series 54 shown in FIG. 14A. The loops 89 are located distally to the loops 87, and are carried at a smaller diameter on the hub 54. Thus, the wires 44 may be guided as desired and may be further protected.

Alternatively, the electrical strip 70 may be rolled in an opposite manner, so that the cable wires 68 and the wires 44 are attached on an inner diameter of the rolled strip 70, instead of its outer diameter. This configuration may allow an additional containment, like a sandwich, of the cable wires 68 and wires 44. Still alternatively, the conductive tracings 72 may be located on both sides of the electric strip 70, with some of the cable wires 68 and wires 44 attached on the inner diameter (as rolled), and some of the cable wires 68 and wires attached on the outer diameter (as rolled). This may allow more electrical connections to fit into a smaller profile. The electrodes 14 of the elongate medical device 2 shown having a linear form in FIG. 1 may sufficiently contact the target body tissue, as body ducts often tend to be serpentine in path, and/or the mucus membranes often withdraw inward, such that at least one or at least two of the electrodes 14 are in contact with the target body tissue, allowing at least some data to be received or recorded at all or most instances.

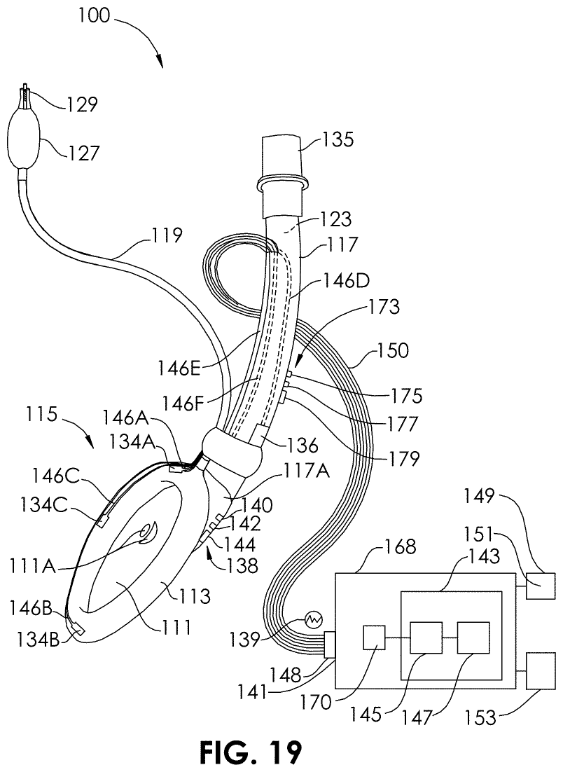

A large range of medical devices having diagnostic and/or therapeutic functionalities may be manufactured as the elongate medical device 2 having features described herein. Some particular examples follow. A system for measurement of cardiovascular parameters 100 is illustrated in FIG. 19. The system for measurement of cardiovascular parameters 100 includes a sensing device which is a laryngeal mask or laryngeal airway (LMA) 115 having sensing capabilities. One method to maintain an oral airway during anesthetic management or mechanical ventilation, utilizes a laryngoscope for endotracheal intubation. Alternatively, a laryngeal mask airway can be inserted into the larynx. A laryngeal mask or laryngeal mask airway (LMA) 115, as shown in FIG. 19, comprises an oval mask body 111 and a hollow cuff 113 which engages the periphery of the mask body 111 and has a ring-shaped luminal area. The hollow cuff 113 may follow the oval shape of the mask body 111. A respiratory tube 117 is connected to a tube connecting portion 117A on the outside surface of the mask body 111. The respiration is performed through the holes 111A which are formed in the mask body 111, and through an elongate passageway 123 in the respiratory tube 117. A fitting 135 is sealingly attached to the respiratory tube 117 and is configured for coupling to mechanical ventilation equipment. The fitting 135 is configured to couple to a respiratory or other oxygen or air delivery apparatus, for delivering oxygen and other gases, which may in some cases include an anesthetic, through the respiratory passageway 123 and out the holes 111A and then into the patient's lungs. An inflation tube 119, fluidly coupled to the cuff 113, is configured for injecting air into the cuff 113. A valve 127 carried in fluid communication with the inflation tube 119 may be used to maintain the pressurized air within the cuff 113. In some embodiments, the valve 127 may be a one-way valve (open or closed). In some embodiments, the valve 127 may be a pinch valve, which is normally in a closed condition be may be pinched to allow air to enter or exit the inflation tube 119. In some embodiments, the valve 127 may be a luer-activated valve which allows air to enter of exit the inflation tube 119 when a luer or a syringe (not shown) is attached to a luer connector 129 at the end of the inflation tube 119. Prior to insertion of the LMA 115, an anesthesiologist or other medical professional deflates the cuff 113 by extracting air therefrom. Once the anesthesiologist or other medical professional inserts the LMA 115 into a patient's larynx, he or she then inflates the cuff 113 by introducing air therein. In this manner, an airway is maintained by covering the larynx with the LMA 115.

The LMA 115 incorporates one or more sensors, which may include one or more cuff-based sensors 134 (134A, 134B, 134C), and one or more tube-based sensors 136. The number of sensors 134, 136 on the cuff 113 and/or the tube 117 (which may include the tube connecting portion 117A) may be varied in different embodiments. In addition, an optical sensor 138 (for example, a pulsed oximetry device) having at least two light emitting sources 140, 142 and one light detector 144, is mounted on the mask body 111 and/or the tube 117/tube connecting portion 117A (shown on the tube connecting portion 117A in FIG. 19). The optical sensor 138 may even be located on the cuff 113, for example, a rearwardly-facing portion of the cuff 113 that does not directly engage tissue of the body lumen when the cuff 113 is inflated. The optical sensor 138 is configured to obtain plethysmographic data when it is positioned in spaced relation with tissue, for example, in a non-contact arrangement with an inner wall of a body lumen. The sensors 134, 136 may comprise electrodes and utilize bio-impedance to generate waveforms representative of the flow of blood through the carotid arteries. Examples of bioelectrical impedance analysis of blood flow using electrode sensors arrayed within body lumens, at least some of the sensors contacting mucosal tissue can be found in U.S. Pat. No. 5,791,349, issued on Aug. 11, 1998, and entitled “APPARATUS AND METHOD OF BIOELECTRICAL IMPEDANCE ANALYSIS OF BLOOD FLOW,” U.S. Pat. No. 5,782,774, issued on Jul. 21, 1998, and entitled “APPARATUS AND METHOD OF BIOELECTRICAL IMPEDANCE ANALYSIS OF BLOOD FLOW,” U.S. Pat. No. 6,095,987, issued on Aug. 1, 2000, and entitled “APPARATUS AND METHODS OF BIOELECTRICAL IMPEDANCE ANALYSIS OF BLOOD FLOW,” U.S. Pat. No. 6,292,689, issued on Sep. 18, 2001, and entitled “APPARATUS AND METHODS OF BIOELECTRICAL IMPEDANCE ANALYSIS OF BLOOD FLOW,” all of which are hereby incorporated by reference in their entirety for all purposes. Electrodes may comprise a coil, a copper band, a gold band, or a silver band. Electrodes may be soldered, welded, crimped, or attached via other mechanical methods.

The sensors 134, 136 are electrically coupled to conductive traces 146A, 146B, 146C, all of which may be painted, sprayed, or printed on the cuff 113, the tube 117, or even the inflation tube 119 by any of the methods and using any of the materials described herein, or by the methods described in international publication number WO2016/179563, published on Nov. 10, 2016, and entitled “SYSTEMS AND METHODS FOR INTERNAL ECG ACQUISITION,” and described in U.S. Pat. No. 9,289,141, issued on Mar. 22, 2016, and entitled “APPARATUS AND METHODS FOR THE MEASUREMENT OF CARDIAC OUTPUT,” which are hereby incorporated by reference in their entirety for all purposes. As shown in FIG. 19, the conductive traces 146A, 146B, 146C are applied onto the cuff 113. Additional conductive traces 146D, 146E, 146F are applied on and within the tube 117 using the materials and methods described in U.S. patent application publication number 2017/0231572, published Aug. 17, 2017, and entitled “SYSTEMS AND METHODS FOR OBTAINING CARDIOVASCULAR PARAMETERS,” which is hereby incorporated by reference in its entirety for all purposes. The conductive traces 146A, 146B, 146C, 146D, 146E, 146F connect the sensors 134, 136 (e.g., electrodes), and optical sensor 138 to a multi-contact connector 148 via an extension 150 which may contain conductive wires or traces. Conductive trace 146D within a portion of the tube 117 connects sensor 136 to the extension 150. Conductive trace 146E within a portion of the tube 117 connects optical sensor 138 to the extension 150. Conductive trace 146F on an external portion of the tube 117 connects conductive trace 146B to the extension 150. Electrical connections between components may be created using solder or mechanical attachment.

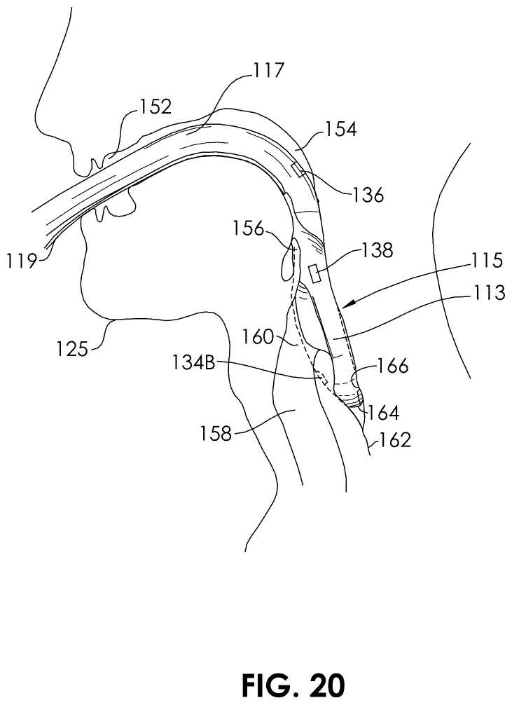

The connector 148 may be configured to be coupled to an input 141 of a console 168 and is configured to carry signals 139 from the one or more sensors 134, 136 and first optical sensor 138 to the console 168. In some embodiments, the console 168 may include an analog-to-digital converter 170 through which the one or more signals 139 are converted. In some embodiments, the signals 139 may be multiplexed. The one or more signals 139 may enter a processor 143 provided by the console 168. The processor 143 may include one or more amplifiers 145 for amplifying the signal 139 and one or more filters 147 for filtering the signal 139. A display 149 is configured to display a resulting graphic representation 151. The graphic representation 151 may simply be a parameter value or a table of values, or may actually be a graph of data, for example a plethysmograph. The display 149 may be built in to the console 168 or may be separate. The display 149 may be directly connected to the console 168 or may be remote and communicate wirelessly. The console 168 may include an interface 153 which allows a user to control and/or communicate with the console 168 or the system for measurement of cardiovascular parameters 100 in general. The interface 153 may even allow a user to control or communicate with the LMA 115, for example, if the LMA 115 incorporates an internal microprocessor, which may be carried on a flex circuit. The interface 153 may be a touch screen, a keyboard, an audio communication system (e.g., voice-activated), and may incorporate a graphic user interface (GUI). The processor 143 is configured to calculate one or more value, including but not limited to, stroke volume, heart rate, and SpO2 from photoplethysmographic data provided by the first optical sensor 138 and the electrocardiogram signal and blood flow information provided by the first, second, and third sensors 134A, 134B, 134C. The emitters 140, 142 and detector 144 of the first optical sensor 138 function as a pulse oximetry device to obtain a photoplethysmograph from the throat or oral cavity by the transmission of optical radiation into a tissue site (tissue at the wall of the throat 154 (FIG. 20), adjacent or at the same level as the carotid arteries), and the detection of the intensity of the optical radiation after absorption by pulsatile blood flow within the tissue site. All three signals (waveforms representative of blood flow, electrocardiogram signal, photoplethysmograph) are utilized to calculate the stroke volume, heart rate, and SpO2 (peripheral capillary oxygen saturation) and to obtain waveforms representative of the arterial flow of central vessels which in this example are one or more of the carotid arteries, but may alternatively be other blood vessels. Cardiac output (CO) is calculated by multiplying stroke volume (SV) by heart rate (HR). When coupled with the values provided by an external blood pressure cuff, real time estimates of arterial blood pressure can also be obtained. Stroke volume variation (SVV) may be determined using methods described in U.S. patent application publication number 2017/023157 to Lowery, published Aug. 17, 2017, and entitled “SYSTEMS AND METHODS FOR OBTAINING CARDIOVASCULAR PARAMETERS,” which is hereby incorporated by reference in its entirety for all purposes.

Turning to FIG. 20, in use, an anesthesiologist or other medical professional positions the LMA 115 so that it covers the larynx 158 of a patient 125. A number of insertion and placement methods may be used. The LMA 115 is shown in FIG. 20 inserted through the mouth 152 of the patient 125 and in place within the throat 154 of the patient 125. The distal end 164 of the LMA 115 is shown adjacent the base 166 of the throat 154, with the cuff 113 shown in relation to the epiglottis 156 and the larynx 158, including the inlet 160 of the larynx. The esophagus 162 is also shown for reference purposes. In some alternative embodiments, a second optical sensor 173 having two light emitting sources 175, 177 and one light detector 179 may be located on a distal portion, or on a more centrally-located portion (as shown in FIG. 4) of the LMA 115, and may be used in conjunction with sensors that are internally located, to allow for the calculation of cardiac output, stroke volume variation and/or other cardiac metrics.

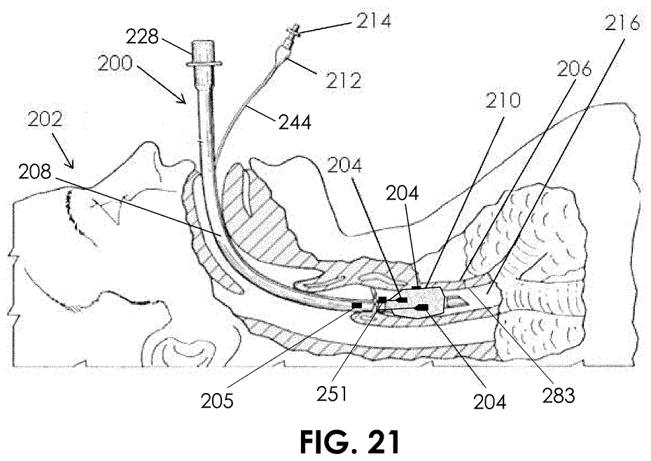

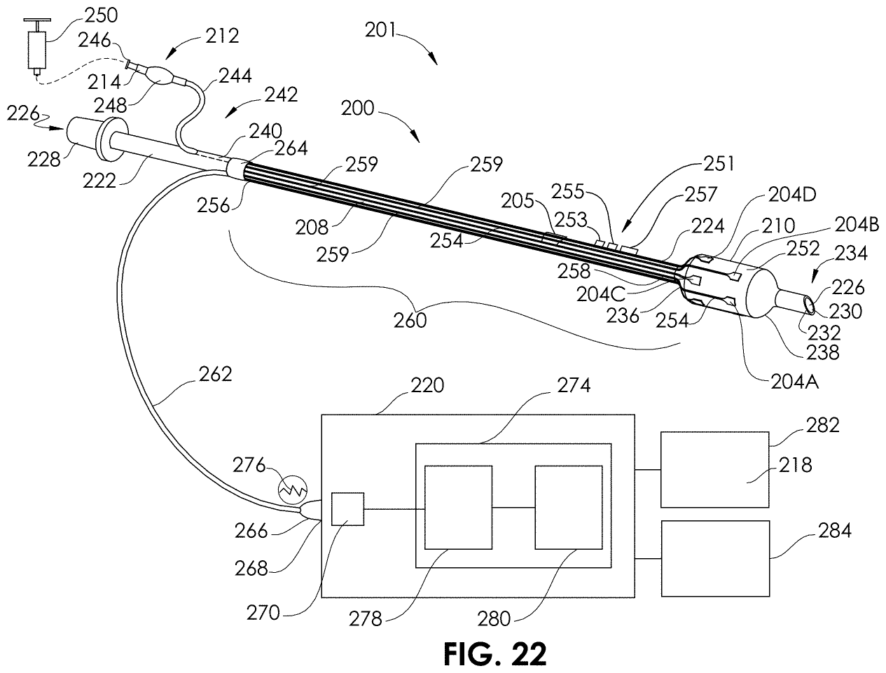

In another embodiment, illustrated in FIG. 21, a sensing device 200 is configured for placement in the lumen 216 of a trachea 206 within a patient 202. The sensing device 200 has the functionality of a trachea tube and includes an elongate member 208 and an actuation portion 210 configured to be expanded within the trachea 206. The sensing device 200 is part of a system for measuring cardiovascular parameters 201, which is shown in more detail in FIG. 22. The system for measuring cardiovascular parameters 201 includes a console 220 to which the sensing device 200 may be coupled. The system for measurement of cardiovascular parameters 201 is configured to sense signals related to cardiovascular parameters of the heart. An elongate member 208 of the sensing device 200 may comprise a shaft or catheter tubing. The elongate member 208 has a proximal end 222 and a distal end 224. The sensing device 200 as depicted in FIG. 22 is configured to serve as an endo-tracheal tube, and thus the sensing device 200 comprises a respiratory lumen 226 extending between a fitting 228, coupled to the proximal end 222 of the elongate member 208 and a port 230 adjacent the distal end 224 of the elongate member 208. The respiratory lumen 226 may be configured to allow the passage of a guidewire (not shown), which may be placed through the respiratory lumen 226 to aid in the delivery of the sensing device 200 within the body cavities of the subject, and which may be subsequently removed. At the port 230, the elongate member 208 may include a skive 232, or angled cut or formed tip, to aid in the tracking of the distal end 234 of the sensing device 200. The fitting 228 is configured to couple to a respiratory or other oxygen or air delivery apparatus, for delivering oxygen and other gases, which may in some cases include an anesthetic, through the respiratory lumen 226 and out the port 230 and into the patient's lungs, for example via the trachea and/or bronchi.

An actuation portion 210 having a proximal end 236 and a distal end 238 is carried by the distal end 224 of the elongate member 208, or may be actually formed from the distal end 224 of the elongate member 208. The actuation portion 210 may comprise an inflatable member, such as a balloon or cuff, or an otherwise expandable structure, and can be configured to have a low-profile state for placement into a body lumen or cavity and delivery within the body lumen or cavity (or within the lumen of a sheath or tube, including a catheter tube). The inflatable member and the elongate member 208 may comprise a polymer such as polyvinyl chloride (PVC) or polyethylene. The actuation portion 210 can also have an expanded state. If the actuation portion 210 is an inflatable member, then the expanded state may be achieved by inflating the actuation portion 210 (inflatable member) with a fluid, such as a gas or liquid. The fluid may include, for example, water, normal saline, air, nitrogen, or other inflation media. An inflation lumen 240 extends from a proximal location 242 to the actuation portion 210 (inflatable member) and is accessed at an interface 212, which may be coupled to the inflation lumen 240 via extension tubing 244. The interface 212 may comprise a luer fitting 246 configured to attach to a syringe or other type of inflation device 250. The interface 212 may include a valve 214, such as a luer-activated valve. The luer-activated valve may be configured to be in a closed (sealed) state when no inflation device is attached to the luer fitting 246, and may be configured to be in an open (unsealed) state when an inflation device is attached to the luer fitting 246. A pilot balloon 248 may be carried on the interface 212 to give tactile or visual feedback for a user to determine the extent of inflation of the inflatable member.

In FIG. 22, the actuation portion 210 is an inflatable member which carries one of more sensors 204 (204A, 204B, 204C, 204D) on its surface 252. Additionally, one or more shaft-based sensors 205 are carried on the elongate member 208. The total number of sensors 204 carried on the actuation portion and sensors 205 carried on the elongate member 208 may be varied in different embodiments. The one or more sensors 204 are secured to the surface 252 of the actuation portion 210 by adhesive or epoxy, or the one of more sensors 204 may be deposited, painted, coated, sprayed, sputtered, or otherwise attached or adhered to the surface 252, as described herein. In some embodiments, the one or more sensors 204 may be applied to the surface 252 of the actuation portion 210 by use of a masking process described herein. In other embodiments, the one or more sensors 204 may be applied by a computer-controlled or robotic applicator which applies the sensor 204 in a computer-controlled pattern to the surface 252. In some embodiments, the one or more sensors 204, 205 are electrodes comprising an electrically conductive material, which may comprise silver, such as a conductive silver ink, carbon ink, a silver-silver chloride ink, or a silver-carbon-silver chloride ink. In some embodiments, a radiopaque ink may be applied along with or adjacent the electrically conductive inks, or may even be the electrically conductive ink. The radiopaque ink increases the ability, for example, to visualize the one or more sensors 204, 205 under radiography or fluoroscopy. Returning to FIG. 21, the valve 214 maintains the desired inflated pressure, and thus maintains the contact of the sensors 204 with the interior wall 283 of the trachea 206.

One or more optical sensors 251, each comprising at least two light emitting sources 253, 255 and one light detector 257, are carried on the elongate member 208. As in the system for measurement of cardiovascular parameters 100 of FIG. 19, the optical sensor 251 is configured to obtain plethysmographic data when it is positioned in spaced relation with tissue, for example, in a non-contact arrangement with an inner wall of a body lumen. Also, as in the system for measurement of cardiovascular parameters 100 of FIG. 19, the sensors 204 utilize bio-impedance to generate waveforms representative of the pulsatile flow of blood. However, because the actuation portion 210 is configured to be placed in the trachea, the adjacent area having significant pulsatile blood flow is the ascending aorta or central vasculature. The ascending aorta represents blood flow close to that of the cardiac output; Doppler methods often rely on the descending aorta for measurements of stroke volume, which does not include flow from the head and upper body portions.

Though the actuation portion 210 is configured to be expanded within the trachea 206, in alternative embodiments, the sensing device 200 may be placed inside the esophagus of a subject, and the actuation portion 210 expanded such that the sensors 204 contact an interior wall of the esophagus. In keeping with the teachings of this disclosure, one or more electrically-conductive tracings 259, each having a proximal end 256 and a distal end 258, are carried upon internally-facing surfaces and/or externally-facing surfaces of the elongate member 208. Electrically-conductive tracings 254 carried on the surface 252 of the actuation portion 210 connect the sensors 204A-D with the electrically-conductive tracings 259. One or more electrically-conductive tracings 259 connect the one or more optical sensors 251 and the one or more sensors 204, 205 (with or without the use of intermediate electrically-conductive tracings 254) to a cable 262, which terminates in a connector 266 which is configured to be coupled to an input 268 of a console 220. A dielectric layer 260 is subsequently applied, where necessary, over the one or more electrically-conductive tracings 259 or electrically-conductive tracings 254. The dielectric materials described herein may include polyimide, adhesive, epoxy, polyethylene shrink tubing, or polyester shrink tubing.

Signals 276 entering the console 220 may in some embodiments represent several different sensors 204, 205, 251 (having been carried by several corresponding electrically-conductive tracings 259, 254). In some embodiments, the console 220 may include an analog-to-digital converter 270 through which the one or more signals 276 are converted. In some embodiments, the signals 276 may be multiplexed. The one or more signals 276 may enter a processor 274 provided by the console 220. The processor 274 may include one or more amplifiers 278 for amplifying the signal 276 and one or more filters 280 for filtering the signal 276. A display 282 is configured to display a resulting graphic representation 218. The graphic representation 218 may simply be a parameter value or a table of values, or may actually be a graph of data. The display 282 may be built in to the console 220 or may be separate. The display 282 may be directly connected to the console 220 or may be remote and communicate wirelessly. The console 220 may include an interface 284 which allows a user to control and/or communicate with the console 220 or the system for measurement of cardiovascular parameters in general. The interface 284 may even allow a user to control or communicate with the sensing device 200, for example, if the sensing device 200 incorporates an internal microprocessor, which may be carried on a flex circuit. The interface 284 may be a touch screen, a keyboard, an audio communication system (e.g., voice-activated), and may incorporate a graphic user interface (GUI).

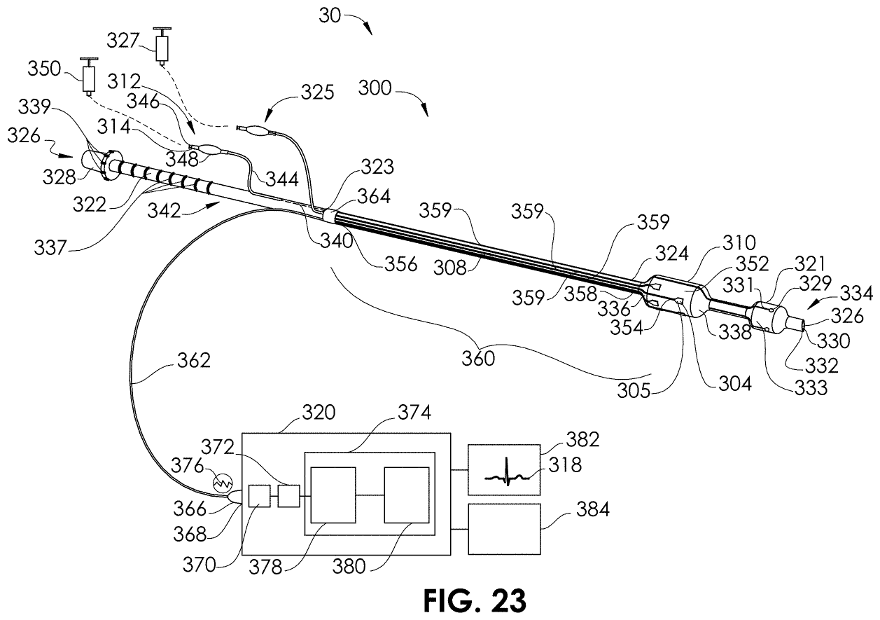

FIG. 23 illustrates a sensing system 30 comprising a sensing device 300 which is configured to be coupled to a console 320. The sensing system 30 is configured to sense signals from the interior of a subject. Such signals may result from bio-impedance, as previously described. Additionally, or alternatively, the signals may include signals related to electrical activity of the heart, such as can be acquired to provide an electrocardiogram. The sensing device 300 comprises an elongate member 308, which may comprise a shaft or catheter tubing. The elongate member 308 has a proximal end 322 and a distal end 324. The sensing device 300 as depicted in FIG. 23 is configured to serve as an endo-tracheal tube having sub-selective capability, and thus the sensing device 300 comprises a respiratory lumen 326 extending between a fitting 328, coupled to the proximal end 322 of the elongate member 308 and a port 330 adjacent the distal end 324 of the elongate member 308. The respiratory lumen 326 may be configured to allow the passage of a guidewire (not shown), which may be placed through the respiratory lumen 326 to aid in the delivery of the sensing device 300 within the body cavities of the subject, and which may be subsequently removed. At the port 330, the elongate member 308 may include a skive 332, or angled cut or form, to aid in the tracking of the distal end 334 of the sensing device 300. The fitting 328 is configured to couple to a respiratory or other oxygen or air delivery apparatus, for delivering oxygen and other gases, which may in some cases include an anesthetic, through the respiratory lumen 326 and out the port 330 in into the patient's lungs, for example via one or more bronchi.

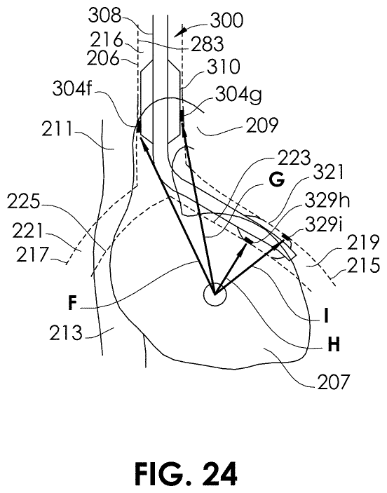

A first actuation portion 310 having a proximal end 336 and a distal end 338 is carried by the distal end 324 of the elongate member 308, or may be actually formed from the distal end 324 of the elongate member 308. The first actuation portion 310 may comprise an inflatable member, such as a balloon or cuff, or an otherwise expandable structure, and can be configured to have a low-profile state for placement into a body lumen or cavity and delivery within the body lumen or cavity (or within the lumen of a sheath or tube, including a catheter tube). The first actuation portion 310 can also have an expanded state. If the first actuation portion 310 is an inflatable member, then the expanded state may be achieved by inflating the first actuation portion 310 (inflatable member) with a fluid, such as a gas or liquid. The fluid may include, for example, water, normal saline, air, nitrogen, or other inflation media. An inflation lumen 340 extends from a proximal location 342 to the first actuation portion 310 (inflatable member) and is accessed at an interface 312, which may be coupled to the inflation lumen 340 via extension tubing 344. The interface 312 may comprises a luer fitting 346 configured to attach to a syringe or other type of inflation device 350. The interface 312 may include a valve 314, such as a luer-activated valve. The luer-activated valve may be configured to be in a closed (sealed) state when no inflation device is attached to the luer fitting 346, and may be configured to be in an open (unsealed) state when an inflation device is attached to the luer fitting 346. A pilot balloon 348 may be carried on the interface 312 to give tactile or visual feedback for a user to determine the extent of inflation of the inflatable member. Distal to the first actuation portion 310 is a second actuation portion 321 which is expandable. The second actuation portion 321 may be an inflatable member, such as a balloon or cuff, and may be expandable through the same inflation lumen 340 as the first actuation member 310, or, as illustrated, may be independently expandable through a second inflation lumen 323 via a second interface 325, which may have similar features to the interface 312. For example, the second interface 325 may be inflated by an inflation device 327. In some embodiments, the first actuation member 310 may be configured to be inflated within a trachea 206 (FIG. 24) while the second actuation portion 321 may be configured to be inflated within a bronchus 215, 217. In some embodiments, the first actuation portion 310 has a larger profile or diameter than the second actuation portion 321. For example, the diameter of the first actuation portion 310 may be between about 5 mm and about 30 mm, or between about 13 mm and about 27 mm, while the diameter of the second actuation portion 321 may be between about 4 mm and 20 mm, or between about 9 mm and about 18 mm.

In FIG. 23, the first actuation portion 310 is an inflatable member which carries one of more sensors 304 on its surface 352. The one or more sensors 304 may be secured to the surface 352 of the first actuation portion 310 by adhesive or epoxy, or the one of more sensors 304 may be deposited, painted, coated, sprayed, sputtered, or otherwise attached or adhered to the surface 352, as described herein. In some embodiments, the one or more sensors 304 may be applied to the surface 352 of the first actuation portion 310 by use of a masking process described herein. In other embodiments, the one or more sensors 304 may be applied by a computer-controlled or robotic applicator which applies the sensor 304 in a computer-controlled pattern to the surface 352. In some embodiments, the one or more sensors 304 are electrodes comprising an electrically conductive material, which may comprise silver, such as a conductive silver ink, carbon ink, a silver-silver chloride ink, or a silver-carbon-silver chloride ink. In some embodiments, a radiopaque ink may be applied along with or adjacent the electrically conductive inks, or may even be the electrically conductive ink. The radiopaque ink increases the ability, for example, to visualize the one or more sensors 304 under radiography or fluoroscopy.

The one or more sensors each have a contact surface 305. Each of the one or more sensors 304 may be coupled to a conductor 354. One or more electrically-conductive tracings 359 are applied to internally-facing surfaces and/or externally-facing surfaces of the elongate member 308, each of the one or more electrically-conductive tracings 359 having a proximal end 356 and a distal end 358. In some embodiments, the one or more sensors 304 and/or the one or more conductors 354, 359 may be applied using methods described in U.S. Pat. No. 9,289,141 entitled “Apparatus and Methods for the Measurement of Cardiac Output,” issued Mar. 22, 2016. The one or more conductors 354 or one or more electrically-conductive tracings 359 may be applied at the same time as the one or more sensors 304 or may be applied before or after the application of the one or more sensors 304. In some embodiments, the one or more sensors 304 are partially applied (e.g., a single layer or a first number of layers), the one or more conductors 354 or one or more electrically-conductive tracings 359 are then applied, and then a final one or more layers are applied to complete the one or more sensors 304. In some embodiments, a dielectric layer 360 is subsequently applied over the one or more electrically-conductive tracings 359 after their application. One or more sensors 329 and one or more conductors 331 are applied to a surface 333 of the second actuation portion 321 by any of the methods described. The one or more conductors 354, 331 may also be coated or otherwise covered by a dielectric material. The one or more electrically-conductive tracings 359 couple the sensors 304, 329 and/or the conductors 331, 354 to a cable 362 (for example, with solder), and a covering or strain relief 364 may be secured over the area of connection. The covering or strain relief 364 may be a dielectric material, including polyimide, adhesive or epoxy, polyethylene or polyester shrink tubing or other similar materials or combinations thereof.

The cable 362 includes a connector 366 which is configured to be coupled to an input 368 of the console 320 and is configured to carry signals 376 from the one or more sensors 304 and/or one or more sensors 329 to the console 320. Signals 376 entering the console 320 may in some embodiments represent several different sensors 304, 329 (having been carried by several corresponding conductors 354, 331 and electrically-conductive tracings 359). In some embodiments, the console 320 may include a lead selector 370 to allow selection of a signal 376 from a particular one of the one or more sensors 304, 329. In some embodiments, one or more signals 376 from one or more sensors 304, 329 may be processed in parallel. The console 320 may include a protection circuit 372, which may include a circuit breaker or other circuit protection device. The one or more signals 376 may enter a processor 374 provided by the console 320. The processor 374 in some embodiments includes one or more amplifiers 378 for amplifying the signal 376 and one or more filters 380 for filtering the signal 376. A display 382 is configured to display a resulting electrocardiogram signal 318 or trace (e.g., PQRST waveform) from the console 320. The display 382 may be built in to the console 320 or may be separate. The display 382 may be directly connect to the console 320 or may be remote and communicate wirelessly. The console 320 may include an interface 384 which allows a user to control and/or communicate with the console 320 or the sensing system 30 in general. The interface may even allow a user to control or communicate with the sensing device 300, for example, if the sensing device 300 incorporates an internal microprocessor, which may be carried on a flex circuit. The interface 384 may be a touch screen, a keyboard, an audio communication system (e.g., voice-activated), and may incorporate a graphic user interface (GUI).

Depth markings 337 and rotational reference markings 339 allow a user to determine the longitudinal and rotational orientation of the sensing device 300 by sight, at the proximal end of the sensing device 300. In some embodiments, an additional sensor may be carried on the second actuation portion 321 which is configured to measure venous oxygenation. The additional sensor may comprise an optical oxygen saturation sensor.

A sensing device 300 is shown in FIG. 24 having sensors 304f, 304g disposed on its first actuation portion 310 which has been located and expanded within the lumen 216 of the trachea 206. In addition, sensors 329h, 329i are disposed on the second actuation portion 321 of the sensing device 300, and the second actuation portion 321 has been located and expanded within a lumen 219 of left bronchus 215. Each of the sensors 304f, 304g are contacting the interior wall 283 of the trachea 206, thus being electrodes for a lead F and lead G, respectively. A first vector F indicates lead F and vector G indicates lead G. Each of the sensors 329h, 329i are contacting an interior wall 223 of the left bronchus 215, thus being electrodes for a lead H and lead I, respectively. Vector H indicates lead H and vector I indicates lead I. Alternatively, the second actuation portion 321 of the sensing device 300 may be tracked into the lumen 221 of right bronchus 217 so that sensors 329 carried on the second actuation portion 321 are able to contact an interior wall 225 of the right bronchus 217. In order to obtain differently-oriented vectors. For orientational reference purposes, the heart 207, the aorta 209, the superior vena cava 211, and the inferior vena cava 213 of the patient are illustrated. Methods and apparatus for acquiring ECG signals are described in U.S. Patent Application Publication Number 2018/0279955 to Lowery, entitled “SYSTEMS AND METHODS FOR INTERNAL ECG ACQUISITION,” published Oct. 4, 2018.

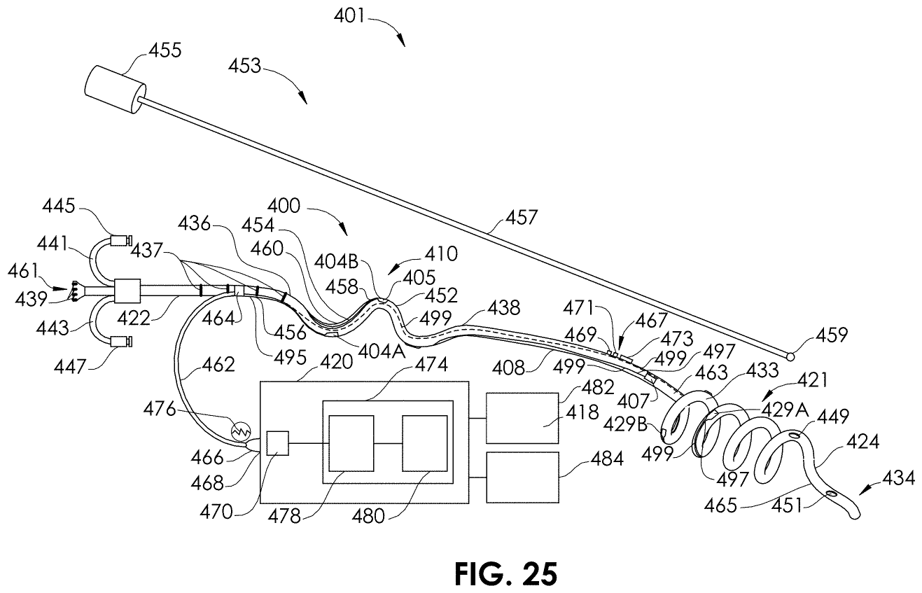

FIG. 25 illustrates a system for measurement of cardiovascular parameters 401 comprising a sensing device 400 which is configured to be coupled to a console 420. The sensing system 401 is configured to sense signals related to cardiovascular parameters of the heart, and may be specifically configured for internally obtaining ECG information. The sensing device 400 comprises an elongate member 408, which may comprise a shaft or catheter tubing. The elongate member 408 has a proximal end 422 and a distal end 424. The sensing device 400 as depicted in FIG. 25 is configured to serve as a nasogastric tube (NG tube), and thus the sensing device 400 comprises one or more lumens 441, 443 extending between one or more fittings 445, 447 coupled to the proximal end 422 of the elongate member 408 and extending through the elongate member until terminating at one or more ports 449, 451 adjacent the distal end 424 of the elongate member 408. One of the ports 449, 451 may be configured for delivery of one or more medicants or for delivery of other fluids (e.g., normal saline) or for delivery of enteral feeding solutions. The ports 449, 451 may be located for direct delivery of the fluids into the stomach, but in alternative embodiments, the sensing device may be configured to allow at least one of the ports 449, 451 to be located in the duodenum or jejunum for direct delivery. In some cases, the port 449, 451 may be located in the distal esophagus. In some embodiments, one of the lumens 441, 443 may be dedicated to fluid delivery while the other lumen 441, 443 is dedicated to suction or lavage of internal contents, for example, stomach contents. In some embodiments, both of the lumens 441, 443 are capable of both delivery and suction or lavage. In some embodiments, the fittings 445, 447 comprises luer fittings, configured to couple to luer fittings of various delivery or suction devices.

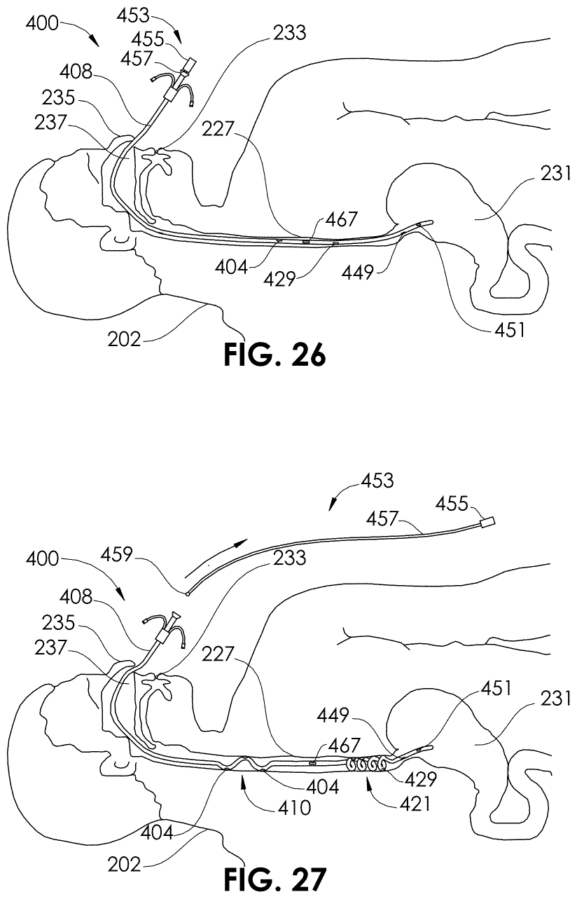

A first actuation portion 410 having a proximal end 436 and a distal end 438 is carried by the elongate member 408. As illustrated in FIG. 25, the first actuation portion 410 in this particular embodiment comprises a secondary shape having an enlarged profile (in comparison to the diameter of the elongate member 408 shaft). The secondary shape is illustrated in FIG. 25 as a serpentine shape or S-shape formed directly in the elongate member 408. The shape may be formed by heat forming of a thermoplastic tubing. A stylet 453 having a proximal hub 455 and an elongate body 457 having a rounded or otherwise blunt tip 459 is configured to be placed down a central lumen 461 of the elongate member 408 of the sensing device 400. FIG. 26 illustrates the sensing device 400 in use, with the elongate body 457 of the stylet 453 inserted within the central lumen 461, causing the first actuation portion 410 to assume a linear or substantially linear orientation, to aid in delivery or movement within a body cavity or lumen. When the sensing device 400 has been delivered to a desired location in the body lumen, for example, the esophagus and stomach, the elongate body 457 of the stylet 453 may be retracted or completely removed from the central lumen 461 of the sensing device 400, to allow the first actuation portion 410 to assume its secondary shape having an enlarged profile (FIG. 27). In other embodiments, the elongate member 408 may comprise a shape memory polymer having shape memory which allows the first actuation portion 410 to achieve its desired secondary shape by contact with a patient's body temperature, or by introduction of a fluid having an increased temperature (e.g., 42° C.) around the elongate member 408. In another alternative embodiment, a shaped shape-memory alloy (e.g., Nitinol) resides within the elongate member 408 and causes the elongate member 408 to change shape at the first actuation portion 410 and/or the second actuation portion 421 when exposed to an elevated temperature (e.g., body temperature or an increased temperature, such as a temperature up to 42° C.). Alternatively, the first actuation portion 410 may be replaced by an inflatable member, such as a balloon or cuff such as those described in prior embodiments herein. In general, the first actuation portion 410 comprises an expandable structure, and can be configured to have a low-profile state for placement into a body lumen or cavity and delivery within the body lumen or cavity (or within the lumen of a sheath or tube, including a catheter tube). As described, the first actuation portion 410 can also have an expanded state.

Distal to the first actuation portion 410 is a second actuation portion 421 having a proximal end 463 and a distal end 465. The second actuation portion 421 is expandable and comprises a low-profile state (FIG. 26) which may be achieved by placement of the elongate body 457 of the stylet 453 through the central lumen 461, and an expanded state (FIGS. 25 and 27) which may be achieved by removal or retraction of the elongate body 457 of the stylet 453 from the central lumen 461. The secondary shape is illustrated in FIGS. 25 and 27 as a spiral or helical shape formed directly in the elongate member 408. Any of the forming materials or methods used in relation to the first actuation portion 410 may also be used in relation to the second actuation portion 421. In some embodiments, the first actuation member 410 may be configured to be expanded within the esophagus while the second actuation portion 421 may be configured to be expanded within the esophagus at a location distal to the first actuation member 410. In some embodiments, the first actuation portion 410 has a smaller profile or diameter than the second actuation portion 421. For example, the (expanded) diameter of the first actuation portion 410 may be between about 15 mm and about 30 mm, or between about 20 mm and about 27 mm, while the (expanded) diameter of the second actuation portion 421 may be between about 25 mm and 40 mm, or between about 30 mm and about 37 mm. In some embodiments, both of the actuation portions 410, 421 may be spiral or helical. In some embodiments, both of the actuation portions 410, 421 may be serpentine or S-shaped. In some embodiments, the first actuation portion 410 may be spiral or helical and the second actuation portion 421 may be serpentine or S-shaped. Other three-dimensional or two-dimensional shapes may be used. In some embodiments, there may only be a single actuation portion, or in other embodiments, there may be three of more actuation portions. Though the ports 449, 451 are shown adjacent a distal end 434 of the sensing device 400, one or more ports 449, 451 may be located some distance proximal to the distal end 434, and in some embodiments proximal to the second actuation portion 421, and in some embodiments, even proximal to the first actuation portion 410. Longitudinal and circumferential markings 437, 439 can be utilized in the sensing device 400 as described in relation to the sensing device 300 of FIG. 23.

In FIG. 25, the first actuation portion 410 carries one of more sensors 404 (404A, 404B) on its outwardly-extending surfaces 452 (e.g., near the outer apex of a curve), such that the one or more sensors 404 are directed against an interior wall of the esophagus (or other body lumen) when the first actuation portion 410 is in its expanded state. Additionally, one or more shaft-based sensors 407 are carried on the elongate member 408. The total number of sensors 404 carried on the actuation portion and sensors 407 carried on the elongate member 408 may be varied in different embodiments. The one or more sensors 404 may be secured to the surface 452 of the first actuation portion 410 by adhesive or epoxy, or the one of more sensors 404 may be deposited, painted, coated, sprayed, sputtered, or otherwise attached or adhered to the surface 452, as described herein. In some embodiments, the one or more sensors 404 may be applied to the surface 452 of the first actuation portion 410 by use of a masking process described herein. In other embodiments, the one or more sensors 404 may be applied by a computer-controlled or robotic applicator which applies the sensor 404 in a computer-controlled pattern to the surface 452. In some embodiments, the one or more sensors 404, 407 are electrodes comprising an electrically conductive material, which may comprise silver, such as a conductive silver ink, carbon ink, a silver-silver chloride ink, or a silver-carbon-silver chloride ink. In some embodiments, a radiopaque ink may be applied along with or adjacent the electrically conductive inks, or may even be the electrically conductive ink. The radiopaque ink increases the ability, for example, to visualize the one or more sensors 404, 407 under radiography or fluoroscopy. In some embodiments, no actuation portion may be necessary, as some ducts, such as the esophagus, are normally collapsed or closed, and naturally contact the elongate member 408, and thus contact the sensors 404, 407 without any actuation. Thus, the first actuation portion 410 and second actuation portion 521 are optional, and the shaft 408 may simply comprise a linear tube.

One or more optical sensors 467, each comprising at least two light emitting sources 469, 471 and one light detector 473, are carried on the elongate member 408. The optical sensor 467 is configured to obtain plethysmographic data when it is positioned in spaced relation with tissue, for example, in a non-contact arrangement with an inner wall of a body lumen. Also, the sensors 404, 429 utilize bio-impedance to generate waveforms representative of the pulsatile flow of blood. Because the actuation portion 410 is configured to be placed in the esophagus, the adjacent area having significant pulsatile blood flow is the ascending aorta.

The sensors 404, 407, 429 are also used to obtain an electrocardiogram signal from the body of the patient to provide electrical timing information, as described in U.S. Patent Application Publication Number 2018/0279955, published Oct. 4, 2018.

The one or more sensors 404 each have a contact surface 405. Each of the one or more sensors 404, 429 or the one or more optical sensors 467 may be coupled to an electrically-conductive tracing 454 having a proximal end 456 and a distal end 458 or electrically-conductive tracing 499 having a proximal end 495 and a distal end 497. One or more electrically-conductive tracings 454, 499 are carried on externally-facing surfaces and/or internally-facing surfaces on or within the elongate member 408. The one or more electrically-conductive tracings 454, 499 may be applied at the same time as the one or more sensors 404, 429 or may be applied before or after the application of the one or more sensors 404. In some embodiments, the one or more sensors 404, 429 are partially applied (e.g., a single layer or a first number of layers), the one or more electrically-conductive tracings 454, 499 are then applied, and then a final one or more layers are applied to complete the one or more sensors 404, 429. In some embodiments, a dielectric layer 460 is subsequently applied over the one or more electrically-conductive tracings 454, 499, as required, after the application of the one or more electrically-conductive tracings 454, 499. One or more sensors 429 (429A, 429B) are applied to outwardly-extending surfaces 433 of the second actuation portion 421 by any of the methods described. Thus, the electrically-conductive tracings 454, 499 are configured to carry signals from the one or more sensors 404, 429, 407 and one or more optical sensors 467 to individual conductors in a cable 462. The cable 462 is electrically coupled to the proximal ends 456, 495 of the one or more electrically-conductive tracings 454, 499 (for example, with solder), and a covering or strain relief 464 may be secured over the area of connection. The covering or strain relief 464 may be a dielectric material, including polyimide, adhesive or epoxy, polyethylene or polyester shrink tubing or other similar materials or combinations thereof. It should be noted that sensing devices 400 having multiple sensors 404, 429, may not require either of the actuation portions 410, 421 in order for the sensors 404, 429 to sufficiently contact certain target body tissue, even in open ducts. For example, the electrodes 14 of the elongate medical device 2 having a linear form in FIG. 1 may sufficiently contact the target body tissue, as the body ducts often tend to be serpentine in path, and/or the mucus membranes often withdraw inward, such that at least one or at least two of the electrodes 14 are in contact with the target body tissue, allowing at least some data to be received or recorded at all or most instances.

The cable 462 includes a connector 466 which is configured to be coupled to an input 468 of the console 420 and is configured to carry signals 476 from the one or more sensors 404, one or more sensors 429, and the one or more optical sensors 467 to the console 420. Signals 476 entering the console 420 may in some embodiments represent several different sensors 404, 429 (having been carried by several corresponding electrically-conductive tracings 454, 499). In some embodiments, the console 420 may include an analog-to-digital converter 470 through which the one or more signals 476 are converted. In some embodiments, the signals 476 may be multiplexed. The one or more signals 476 may enter a processor 474 provided by the console 420. The processor 474 in some embodiments includes one or more amplifiers 478 for amplifying the signal 476 and one or more filters 480 for filtering the signal 476. A display 482 is configured to display a resulting graphic representation 418. The graphic representation 418 may simply be a parameter value or a table of values, or may actually be a graph of data. The display 482 may be built in to the console 420 or may be separate. The display 482 may be directly connected to the console 420 or may be remote and communicate wirelessly. The console 420 may include an interface 484 which allows a user to control and/or communicate with the console 420 or the system for measurement of cardiovascular parameters in general. The interface may even allow a user to control or communicate with the sensing device 400, for example, if the sensing device 400 incorporates an internal microprocessor, which may be carried on a flex circuit. The interface 484 may be a touch screen, a keyboard, an audio communication system (e.g., voice-activated), and may incorporate a graphic user interface (GUI).

The system for measurement of cardiovascular parameters 401 described herein is useful to measure physiological functions/parameters in mammalian subjects, including stroke volume, cardiac output, and stroke volume variation. Once the actuation portion 410, 421 is positioned and expanded, a current is injected into the subject's tissue through one of the electrodes (sensors 404, 429, 407) serving as a current electrode, a voltage is established between the current electrode and the ground electrode (one of sensors 404, 429, 407) so that a current flows through the tissue disposed between the current electrode and the ground electrode. With one or more sense electrodes (sensors 404, 429), the voltages caused by the current flowing in the tissue are detected, wherein the voltages vary in accordance with changes in the bioelectrical impedance of the tissue.

A sensing device 400 is shown in FIG. 26 with the stylet 453 inserted inside the elongate member 408 and being delivered through the nasal cavity 237 of the nose 235 of a patient 202 and into the esophagus 227. The mouth 233 is shown as a reference point. In FIG. 27, the stylet 453 is removed from the sensing device 400 and the elongate member 408 is adjusted (if necessary) so that the first actuation portion 410 and second actuation portion 421 assume their secondary expanded states in their desired locations. The sensors 404, 429 are applied against interior wall portions of the esophagus 227 by the first actuation portion 410 and second actuation portion 421. Port 451 has been placed into the interior of the stomach 231 for fluid delivery, suction, lavage, or other procedural purposes.

The optical sensor 467, when the actuation portion 410 is expanded within the esophagus 227, is in a spaced (non-contact) relation with the interior wall of the esophagus, thus allowing for the reflectance of the optical radiation.

In the embodiments presented in FIGS. 19-27, it should be understood that any of the embodiments presented herein may be utilized as the tube 117 or elongate member 208, 308, 408, depending on the number of electrically-conductive tracings required and/or the orientation of the electrically-conductive tracings that is most efficient. Additionally, other alternatives of the embodiments presented herein are also contemplated which utilize variations of the number of elongate members/tubes or number and orientation of the electrically-conductive tracings. Furthermore, in any of the embodiments presented in FIGS. 19-27, the embodiments of the connectors presented herein, or variations of these embodiments may be utilized, again, depending on the particular requirements of number of electrically-conductive tracings and their orientation.

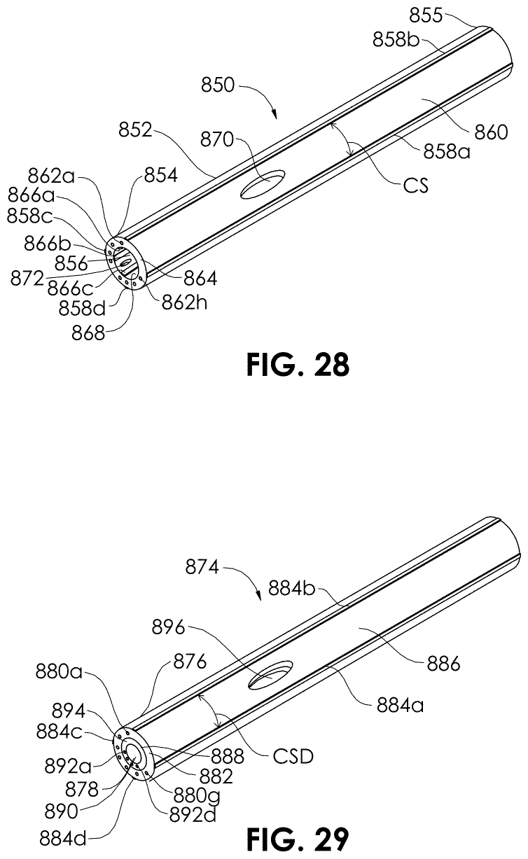

FIG. 28 illustrates an alternative embodiment of an elongate medical device body 850 comprising an elongate member 852 having a lumen 856 extending therethrough. There are eight elongate conductors 862a-h, which may include copper wire, embedded in the wall 864 of the elongate member 852. Alternatively, the conductors 862a-h may be inserted through lumens, or may be injected through lumens as epoxy or adhesive, and cured/solidified. The wire may alternately comprise other conductive materials, such as silver, gold, or platinum. In some special cases, the wire may even comprise less conductive materials such as stainless steel. The elongate conductors may have a diameter of between about 0.004 inches and about 0.012 inches, or about 0.005 inches to about 0.010 inches, or about 0.006 inches to about 0.009 inches. The elongate conductors 862 may include a dielectric coating, but this may not be necessary if the material of the elongate member is an electrically insulative polymer, such as polyvinyl chloride (PVC), or other insulative polymers. Electrically-conductive tracings 866a-c are applied onto an inner surface 868 and electrically-conductive tracings 858a-d are applied onto an outer surface 860 by the methods disclosed herein, thus creating a composite multi-conductor (wires and tracings) system. The combination of wires 862 embedded within the wall 864 of the elongate member 852, electrically-conductive tracings 866 on the inner surface 868, and electrically-conductive tracings 858 on the outer surface 860 allows the incorporation of an even larger number of conductors to carry signals from one end 854 to the other end 855 of the elongate member 852.

Additionally, a first orifice 870 and second orifice 872 can be created in the wall 864 of the elongate member 852 (tubing) without damaging any of the elongate conductors 862 or electrically-conductive tracings 858, 866. As shown in FIG. 28, a circumferential space CS exists between electrically-conductive tracing 858a and electrically-conductive tracing 858b. This circumferential space CS is also between conductor 862a and 862h. Furthermore, the circumferential space CS is between electrically-conductive tracing 866a and electrically-conductive tracing 866c. Thus, there are no electrically-conductive tracings 858, 866 or conductors 862 within the circumferential space CS. Thus, a first orifice 870 is made (e.g., by drilling or cutting) in a single wall thickness of the elongate member 852 to fluidly connect the lumen 856 to the external environment. In order to assure that no electrically-conductive tracings 858, 866 or conductors 862 are damaged upon constructing the first orifice 870, the circumferential orientation of the conductors 862 is controlled during the embedding process (e.g., co-extrusion, over-extrusion, multilayer-extrusion). Additionally, the circumferential orientation of each of the electrically-conductive tracings 858, 866 is controlled with respect to the circumferential orientation of the conductors 862.

The second orifice 872 is made in a single wall thickness of the elongate member 852 to fluidly connect the lumen 856 to the external environment. The second orifice 872 is made by drilling or cutting through the wall 864 between electrically-conductive tracing 858c and electrically-conductive tracing 858d, between conductor 862d and 862e (the conductors shown on either side adjacent to second orifice 872), and between electrically-conductive tracing 866b and electrically-conductive tracing 866c. In order to assure that no electrically-conductive tracings 858, 866 or conductors 862 are damaged upon constructing the second orifice 872, the circumferential orientation of the conductors 862 is controlled during the embedding process (e.g., co-extrusion, over-extrusion, multilayer-extrusion), and the circumferential orientation of each of the electrically-conductive tracings 858, 866 is controlled with respect to the circumferential orientation of the conductors 862. The tracings 858 may incorporated methods and materials described in U.S. Patent Application publication number 20190200930, published Jul. 4, 2019, and entitled “Medical Devices with Layered Conductive Elements and Methods for Manufacturing the Same,” which is hereby incorporated by reference in its entirety for all purposes.

In some embodiments, a shorted portion 891 can be made by cutting or grinding a portion of the wall 864 and soldering, or electrically connecting be conductive epoxy or adhesive, the exposed portion of the conductor 862h to an adjacent portion of the electrically-conductive tracing 858a. The conductor 862h and the electrically conductive tracing 858a now form a longer, two-section conductor that carries a signal in one longitudinal direction and then returns at least a portion of the length of the medical device body 850 in the opposite longitudinal direction. In other embodiments, an electrode may be attached to one of the embedded conductors 862 by removing material from the wall 864, and soldering, crimping or otherwise electrically coupling a metallic band to the exposed portion of the conductor 862. In other embodiments, an electrode may be attached to one of the embedded conductors 862 by removing material from the wall 864, and painting a metallic or metallized material over the exposed portion of the conductor, and around at least a portion of the elongate member 852. Techniques similar to those described in relation to FIGS. 9-10 and 17 may be utilized.

FIG. 29 illustrates an alternative embodiment of a medical device body 874 that, like the medical device body 850 of FIG. 28, includes a composite conductor structure. The medical device body 874 comprises an elongate member 876 having a lumen 878 extending therethrough. There are seven elongate conductors 880a-g, which may include copper wire, embedded in the wall 882 of the elongate member 876. Electrically-conductive tracings 884a-d are applied onto an outer surface 886 of the elongate member 876. The elongate member 876 is coupled to an elongate member 888 having a lumen 890. The elongate member 888 includes four elongate conductors 892a-d embedded in its wall 894. There is a circumferential space CSD between electrically-conductive tracing 884a and electrically-conductive tracing 884b. This circumferential space CSD includes a portion of the space between conductor 880a and conductor 880g, and a portion of the space between conductor 892a and conductor 892d. Thus, an orifice 896 is safely made through the wall 882 of the elongate member 876 and the wall 894 of the elongate member 888, without causing any damage to the conductors 880, 892 or the electrically-conductive tracings 884. The circumferential orientation of the conductors 880 and the conductors 892 is controlled during each of the embedding processes (e.g., co-extrusion, over-extrusion, multilayer-extrusion) of the two elongate members 876, 888, and the circumferential orientation of each of the electrically-conductive tracings 884 is controlled with respect to the circumferential orientation of the conductors 880, 892.

In both the medical device body 850 of FIG. 28 and the medical device body 874 of FIG. 29, the circumferential space CS, CSD may include a sector of the medical device body 850, 874 that is between about 5° and about 180°, or between about 15° and about 120°, or between about 30° and about 90° or the total circumference or perimeter of the medical device body 850, 874. The word “perimeter” is intended to include circular perimeters, but to also include perimeters of cross-sections that are substantially non-circular. In some embodiments, the medical device bodies 850, 874 may also include additional lumens. In some embodiments, the medical device bodies 850, 874 may include additional shafts or tubes within their interior or around their exterior. These additional shafts or tubes may each include their own one or more electrically-conductive tracings or embedded conductors. The embedded conductors in come embodiments in the medical device bodies 850, 874 may extend through lumens. The electrically-conductive tracings and/or the conductors may extend substantially longitudinally, or may have a non-linear path, such as a serpentine path or a helical path.

The circumferential space CS, CSD is created by alignment of two or more nonuniform, unevenly dispersed arrays, comprising two or more or three or more electrically-conductive tracings and/or conductors. The orifices 870, 896 allow the passage of fluid from an internal lumen 856, 890 to an exterior of the medical device body 850, 874, for example, into tissue or a body lumen or cavity of a patient.

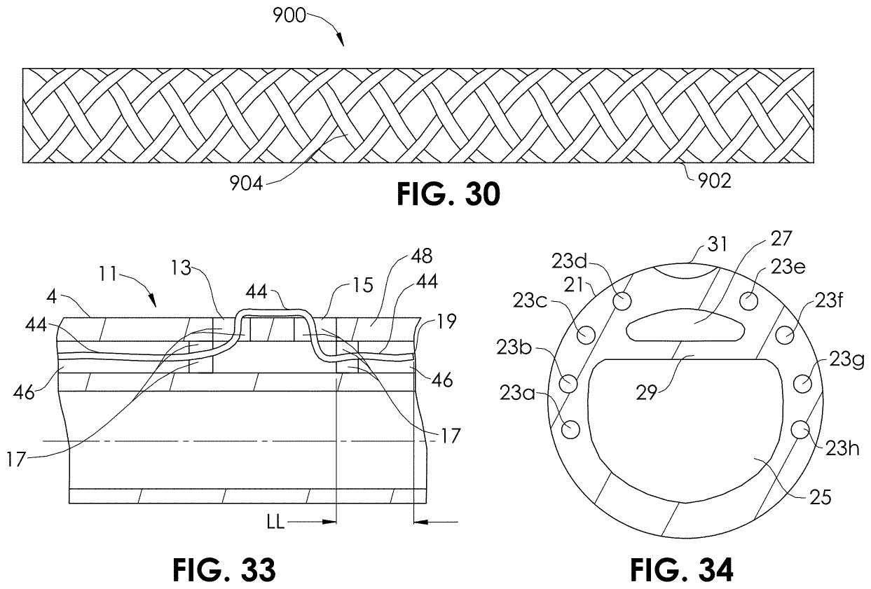

A medical device body 900 is illustrated in FIG. 30 which includes a polymeric tube 902 having one or more embedded spiral-shaped conductors 904. The polymeric tube 902 may be overextruded over the conductors 904, and may comprise any of the materials described herein for tubing, including PVC, polyurethane, or polyether block amide. The conductors 904 may comprise 1, 2, 3, 4, 5, 6, 7, 8, or more copper spiral wires. In one embodiment, a first polymeric extrusion is made in a first step. In a second step, the conductors 904 are wound or braided over the first polymeric extrusion. In a third step, a second extrusion layer is extruded over the first extrusion layer and conductors 904. The first extrusion layer and second extrusion layer at least partially bind to each other creating the polymeric tube 902 portion. In other embodiments, the entire polymeric tube 902 may be extruded together with the conductors 904. In certain embodiments, each of the conductors 904 may have its own dielectric coating or sheath, in order to better assure isolation between the conductors 904 when they are combined into the polymeric tube 902. The conductors 904 may be wound or combined with each other, or may even be braided together, partially or completely. Once the medical device body 900 is formed, one or more of the conductors 904 may be exposed by removing a portion of the polymeric tube 902 covering the conductors 904, in similar manners to those described in relation to FIGS. 9-10 and 17. The conductors 904 may comprise circular or oval cross-section wires, or may comprise flat wire, which may allow for an even lower profile. The spiral orientation of the conductors 904 can allow for a smaller over all profile medical device body 900 to be formed, which can be especially helpful in creating pediatric devices or other devices intended for small ducts and cavities in the body. Though the length of each wire is longer than a wire that is completely longitudinally-extending, and thus the same length as the body 900 itself, the configuration allows for a large number of separate conductors within a relatively thin layer.

The medical device bodies 850, 874, 900 or variations thereof may also be incorporated into any of the medical system embodiments described in FIGS. 19-27. The medical device bodies 850, 874, 900 described herein may be incorporated into a variety of medical devices, and may have a diameter or (if nor circular) a maximum transverse dimension of between about 0.5 mm and about 40 mm, or between about 1 mm and about 30 mm, or between about 2 mm and about 15 mm. As described, the medical devices incorporating the medical device bodies 4, 850, 874, 900 may be configured for performing therapeutic procedures or diagnostic procedures, or for performing both, either at separate periods or at the same time. Though electrically-conductive tracings and elongate conductors are described herein, a similar configuration of conductive tracings and/or elongate wires may be used for more resistive tracings or wires. For example, resistive tracings on an external surface of a medical device body may be used to apply heat, for ablation, or to warm tissue or body or injected fluids, or to increase the activity of a drug. Resistive tracings may also be used to produce light for visualization or measurement. Resistive tracings may be used to measure temperature, for example to control a device from reaching high temperatures that might otherwise damage tissue, such as tissue of the esophagus. Resistive tracings may even be used to pace a heart.

Other medical applications, treatments, procedures, or devices therefor that may benefit from the medical device bodies 4, 850, 874, 900 described herein include, but are not limited to the following: pacing leads; catheters or probes for imaging, including ultrasound, phased-array ultrasound, rotational ultrasound, forward looking ultrasound optical coherence tomography (OCT), infrared, near-infrared, electrophysiology mapping, thermographic imaging, elastographic imaging, catheters or probes for heating or energy delivery, including cancer treatment, sterilization of fallopian tube or other ducts, nerve ablation or therapy, cystic duct ablation, biliary duct ablation, lymph node ablation, urethral cancer treatment, neurovascular energy delivery for closure of aneurysms or arteriovenous malformations (AVMs), closure of heart defects such as patent foramen ovale (PFO), left atrial appendage (LAA), atrial septal defect (ASD), esophageal heating or energy application or ablation including Barrett's esophagus, gastroesophageal reflux disease (GERD), stimulation of reproductive system elements to increase fertility; laser imaging or treatment catheters or probes; heating of shape memory alloy elements in catheters or probes to cause shape change or movement therein; heating of internal elements in catheters or probes to cause shape memory polymers to heat and to change shape or cause motion.

While embodiments have been shown and described, various modifications may be made without departing from the scope of the inventive concepts disclosed herein.

“Externally-facing” is defined as facing toward an outward direction, but is not limited to a most external face. For example, an externally-facing surface may have one or more additional layers of tubing covering it. “Internally-facing” is defined as facing toward an inward direction, but is not limited to a most internal face. For example, an internally-facing surface may have one or more additional layers of tubing inside it.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers (e.g., about 10%=10%), and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

1-37. (canceled)

38. A method for creating a conductive junction in a system for performing a diagnostic or therapeutic procedure, comprising:
placing a first elongate conductor through a first lumen of an elongate body;
creating a hole in a wall of the elongate body adjacent the first lumen at a distal portion of the elongate body;
applying an electrically conductive material to a portion of an outer circumference of the elongate body at the distal portion of the elongate body to form an electrode; and
electrically coupling the first elongate conductor to the electrode.

39. The method of claim 38, wherein the stop of electrically coupling comprises physically connecting the first elongate conductor to the electrode with an electrically conductive epoxy, and electrically conductive adhesive, or an electrically conducive ink.

40-41. (canceled)
